(19) United States
(12) Patent Application Publication
(10) Pub. No.: US 2022/0227875 A1
(43) Pub. Date: Jul. 21, 2022

(54) VECTOR-BASED THERAPY FOR THYROID DISEASE

(71) Applicant: University Of Florida Research Foundation, Incorporated, Gainesville, FL (US)

(72) Inventors: Scott A. Rivkees, Gainesville, FL (US); Arun Srivastava, Gainesville, FL (US)

(73) Assignee: University Of Florida Research Foundation, Incorporated, Gainesville, FL (US)

(21) Appl. No.: 17/615,041

(22) PCT Filed: May 29, 2020
(86) PCT No.: PCT/US2020/035441

§ 371 Date: Nov. 29, 2021

Related U.S. Application Data
(60) Provisional application No. 62/855,865, filed on May 31, 2019.

(51) Int. Cl. C07K 16/28; A61K 35/76; A61K 48/00; A61K 49/00; A61P 5/16

(52) U.S. Cl. CPC C07K 16/2869; A61K 35/76; C07K 2317/56; A61K 49/0002; A61P 5/16; A61K 48/0066

(57) ABSTRACT

Disclosed herein are compositions and methods for continuous AAV-based delivery of blocking anti-TSHR antibodies to a subject having a thyroid disease. The present disclosure is based, at least in part, on the realization that blocking anti-TSHR antibodies may be delivered in a continuous manner using rAAV, e.g., rAAVS, to effectively block the stimulating effects of TSAbs or TSH on TSHR, thereby blocking or reducing the synthesis of thyroid hormone. By blocking or reducing the synthesis of thyroid hormone, the presently described methods and compositions for rAAV-based delivery of blocking anti-TSHR antibodies (e.g., may be used to treat thyroid diseases, including Graves' disease, Graves' orbitopathy, and thyroid cancer without the requirement of repeated administrations.

### BACKGROUND

The present invention relates to methods for treating thyroid disorders caused by hyperthyroidism, e.g., Graves' disease, Graves' orbitopathy, and thyroid cancer. In particular, the present invention relates to methods for treating thyroid disorders by gene-therapy based delivery of blocking anti-TSHR (thyroid-stimulating hormone receptor) antibodies and TSHR action using an expression vector, e.g., an adeno-associated vector, such as, but not limited to, AAV8, to achieve continuous expression in a subject in need thereof.

Thyroid hormones are produced and released by the thyroid gland, namely triiodothyronine (T3) and thyroxine (T4). The thyroid hormones act on nearly every cell in the body and have wide-ranging physiological effects. For instance, thyroid hormones act to increase the basal metabolic rate, regulate growth, and facilitate neural maturation and function. In addition, thyroid hormones are essential to proper development and differentiation of cells of the human body by regulating processes such as protein, fat, and carbohydrate metabolism and the body's use of energetic compounds.

Receptors for thyroid hormones are typically intracellular DNA-binding proteins that function as hormone-responsive transcription factors that regulate responsive genes. The effect of the hormone-receptor complex binding to DNA is to modulate gene expression, either by stimulating or inhibiting transcription of specific genes.

Thyroid-related disease is associated with either inadequate production (i.e., hypothyroidism) or overproduction (i.e., hyperthyroidism) of thyroid hormones. Both types of disease are relatively common afflictions of man and animals. Hyperthyroidism, in particular, results from heightened secretion of thyroid hormones. In most species, this condition is less common than hypothyroidism. In humans the most common form of hyperthyroidism is Graves' disease, an immune disease in which autoantibodies bind to and activate the thyroid-stimulating hormone receptor (TSHR) leading to continual stimulation of thyroid hormone synthesis and release. These types of autoantibodies are also referred to as thyroid-stimulating antibodies (TSAbs).

Common signs of hyperthyroidism include nervousness, insomnia, high heart rate, eye disease and anxiety. Graves' disease is commonly treated with anti-thyroid drugs (e.g., propylthiouracil and methimazole), which suppress synthesis of thyroid hormones primarily by interfering with iodination of thyroglobulin by thyroid peroxidase. Graves' disease is also treated by radioactive iodine or surgery. In addition, thyroid-blocking antibodies (TBAbs) have been identified which block TSHR stimulation by thyroid-stimulating hormone (TSH) or autoantibodies thereby reducing the overall synthesis of thyroid hormone (9, 10). For example, the thyroid-blocking monoclonal antibody K1-70 is under investigation as a treatment for Graves' disease (50).

However, infusion of antibodies, antibody fragments, or peptides that potentially block TSAbs, such as K1-70, last only for a short period of time and require repeated administration (6, 7). Improved methods of delivering clinically significant levels of thyroid-blocking antibodies which would not require repeated administrations would represent a significant advancement in the art for treating thyroid disorders, such as Graves' disease, Graves' orbitopathy, and thyroid cancer.

### SUMMARY

The present disclosure is based, at least in part, on the realization that blocking anti-TSHR antibodies may be delivered in a continuous manner using rAAV, e.g., rAAV8, to effectively block the stimulating effects of TSAbs or TSH on TSHR, thereby blocking or reducing the synthesis of thyroid hormone. By blocking or reducing the synthesis of thyroid hormone, the presently described methods and compositions for rAAV-based delivery of blocking thyroid-stimulating hormone receptor antibodies (anti-TSHR antibodies, e.g., K1-70) may be used to treat thyroid diseases, including Graves' disease, Graves' orbitopathy, and thyroid cancer without the requirement of repeated administrations.

Accordingly, in some aspects, this disclosure provides a recombinant adeno-associated virus (rAAV) particle comprising a nucleic acid molecule that encodes a blocking thyroid-stimulating hormone receptor antibody (anti-TSHR antibody) or fragment thereof. In some embodiments, this disclosure also provides a recombinant adeno-associated virus (rAAV) particle comprising a polynucleotide encoding a blocking anti-TSHR antibody (e.g., K1-70) or functional fragment thereof.

In various embodiments, the blocking anti-TSHR antibody is K1-70 comprising (i) a heavy chain sequence of SEQ ID NO: 1 (Accession No. 2XWT_A, “Chain A, THYROID BLOCKING HUMAN AUTOANTIBODY K1-70 HEAVY CHAIN”):

(SEQ ID NO: 1)
EVQLVQSGAE VKKPGQSLKI SCKASGYSLT DNWIGWVRQK
PGKGLEWMGI IYPGDSDTRY SPSFQGQVTI SADKSINTAY
LQWSSLKASD TAIYYCVGLD WNYNPLRYWG PGTLVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKKVEPK S,

and (ii) a light chain sequence of SEQ ID NO: 2 (Accession No. 2XWT_B, “Chain B, THYROID BLOCKING HUMAN AUTOANTIBODY K1-70 LIGHT CHAIN”):

(SEQ ID NO: 2)
QSVLTQPPSV SAAPGQKVTI SCSGSSSDIG SNYVSWYQQF
PGTAPKLLIY DNNKRPSAIP DRFSGSKSGT SATLGITGLQ
TGDEADYYCG TWDSRLGIAV FGGGTQLTVL GQPKAAPSVT
LFPPSSEELQ ANKATLVCLV SDFYPGAVTV AWKADGSPVK
VGVETTKPSK QSNNKYAASS YLSLTPEQWK SHRSYSCRVT
HEGSTVEKTV APTE.

See Sanders, J. et al., “Human monoclonal thyroid stimulating autoantibody,” Lancet 362 (9378), 126-128 (2003) (which is incorporated herein by reference).

In certain embodiments, this disclosure provides a recombinant adeno-associated virus (rAAV) particle comprising a polynucleotide sequence encoding an anti-TSHR antibody polypeptide having the structure of HSP-VH-CH-F2A-LSP-VL-CL, wherein:

HSP refers to a signal peptide;

VH refers to a human IgG1 heavy chain variable region;

CH refers to a human IgG1 heavy chain constant region;

F2A refers to an F2A cleavage site;

LSP refers to a lambda-1 light chain signal peptide;

VL refers to a human IgG1 light chain variable region; and

CL refers to a human IgG1 light chain constant region.

In certain embodiments, the disclosure provides a recombinant adeno-associated virus (rAAV) particle comprising a polynucleotide sequence encoding an anti-TSHR antibody polypeptide having the structure of HSP-VH-CH-F2A-LSP-VL-CL, wherein:

HSP refers to a signal peptide;

VH refers to a K1-70 heavy chain variable region;

CH refers to a K1-70 heavy chain constant region;

F2A refers to an F2A cleavage site;

LSP refers to a K1-70 light chain signal peptide;

VL refers to a K1-70 light chain variable region; and

CL refers to a K1-70 light chain constant region.

In some embodiments, the nucleic acid molecule comprises a promoter operably linked to an expression cassette. In some embodiments, the polynucleotide comprised in a rAAV particle further comprises a promoter to drive transcription of the anti-TSHR antibody polypeptide. In some embodiments, the promoter is a truncated chimeric CMV-chicken β-actin (smCBA) promoter.

In some embodiments, the expression cassette comprises in a 5′-to-3′ direction a first sequence encoding a heavy chain of the blocking anti-TSHR antibody, a second sequence encoding a self-cleaving site, and a third sequence encoding a light chain of the blocking anti-TSHR antibody. In some embodiments, the expression cassette comprises in a 5′-to-3′ direction a first sequence encoding a light chain of the blocking anti-TSHR antibody, a second sequence encoding a self-cleaving site, and a third sequence encoding a heavy chain of the blocking anti-TSHR antibody.

In some embodiments, the self-cleaving site is F2A cleavage site. In some embodiments, the first sequence and the third sequence are each preceded by a signal sequence. In some embodiments, the nucleic acid molecule further comprises a poly-A tail sequence. In some embodiments, the nucleic acid molecule further comprises an HA sequence. In some embodiments, the nucleic acid molecule further comprises inverted terminal repeat (ITR) sequences at the 5′ and 3′ ends of the expression cassette.

In some embodiments, a rAAV particle is of serotype 1. In other embodiments, the rAAV particle is of serotype 2. In still other embodiments, the rAAV particle is of serotype 3. In yet other embodiments, the rAAV particle is of serotype 4. In some embodiments, a rAAV particle is of serotype 5. In other embodiments, the rAAV particle of serotype 6. In still other embodiments, the rAAV particle is of serotype 7. In yet other embodiments, the rAAV particle is of serotype 8. In some embodiments, a rAAV particle is of serotype 9. In other embodiments, the rAAV particle of serotype 10. In still other embodiments, the rAAV particle is of serotype 11. In some embodiments, a rAAV particle is of serotype 12. In other embodiments, the rAAV particle is of serotype 13. In still other embodiments, the rAAV particle is of serotype 2/1. In yet other embodiments, the rAAV particle is of serotype 2/5 In some embodiments, a rAAV particle is of serotype 2/8. In other embodiments, the rAAV particle is of serotype 2/9. In still other embodiments, the rAAV particle is of serotype 3/1. In yet other embodiments, the rAAV particle is of serotype 3/5. In some embodiments, a rAAV particle is of serotype 3/8. In other embodiments, the rAAV particle is of serotype 3/9.

In a preferred embodiment, the rAAV particle is of serotype 8.

In various embodiments, the rAAV particle may be a self-complementary rAAV particle.

In some embodiments, the blocking anti-TSHR antibody is K1-70. In some embodiments, the K1-70 has a heavy chain of SEQ ID NO: 1 or a sequence having at least 90% sequence identity to SEQ ID NO: 1 and a light chain of SEQ ID NO: 2 or a sequence having at least 90% sequence identity to SEQ ID NO: 2.

In some embodiments, the nucleic acid further encodes a detectable molecule.

In some embodiments, the P2A tag becomes cleaved such that the heavy chain and the light chain are expressed as separate molecules.

In some embodiments, an expression level of the antibody is continuous.

In some aspects, provided herein are compositions comprising a plurality of any one of the rAAV particles as disclosed herein. In some embodiments, a composition comprises a pharmaceutically acceptable carrier.

In other aspects, provided herein are compositions comprising a plurality of any one of the polynucleotides comprised in the rAAV particles as disclosed herein. In some embodiments, a composition comprises a pharmaceutically acceptable carrier.

In some aspects, provided herein are kits comprising any one of the compositions comprising any one of the rAAV particles or the isolated polynucleotides thereof as disclosed herein. In some embodiments, a kit comprises instructions for using the composition comprised in the kit.

In some aspects, provided herein is a method comprising administering any one of the compositions comprising rAAV particles disclosed herein to a subject, wherein the administering comprises an injection of the composition. In some embodiments, an administration of the composition is repeated at least once, and wherein the time between a repeated administration and a previous administration is at least 1 month. In some embodiments, an administration of the composition is repeated at least once, and wherein the time between a repeated administration and a previous administration is at least 2 months. In some embodiments, an administration of the composition is repeated at least once, and wherein the time between a repeated administration and a previous administration is at least 5 years.

In some aspects, provided herein is a method for treating a hyperthyroid disorder, comprising administering an effective amount of the composition to a subject. In some embodiments, a subject is human. In some embodiments, a subject (e.g., a human subject) is suffering from or is at risk for developing a hyperthyroid disease, such as Graves' disease, Graves' orbitopathy, and thyroid cancer.

In some embodiments, the anti-TSHR antibody blocks activation of a thyroid-stimulating hormone receptor (TSHR) by a thyroid-stimulating hormone (TSH) and a thyroid-stimulating antibody (TSAb). In some embodiments, the anti-TSHR antibody inhibits TSH production.

In some aspects, provided herein is a method of blocking the activation of a TSHR cell, comprising a plurality of the rAAV particles and a pharmaceutically acceptable carrier.

In some aspects, provided herein is a method of blocking the activation of a TSHR cell, comprising administering an effective amount of a composition comprising a plurality of the rAAV particles and a pharmaceutically acceptable carrier to a subject. In some embodiments, the subject is human.

In some aspects, provided herein is a use of the rAAV particle for delivering the antibody to the TSHR. In some aspects, provided herein is a use of the rAAV particle for treating or diagnosing a hyperthyroid disorder.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A to 1F shows K1-70 inhibits TSH and M22 activation of the TSHR. (FIG. 1A) cAMP levels rise in HEK-TSHR cells treated with TSH. (FIG. 1B) TBAb K1-70 blocks TSH-induced increases in cAMP. (FIG. 1C) TBAb K1-70 blockage does not alter cAMP in absence of TSH. (FIG. 1D TSAb M22 increases cAMP levels. (FIG. 1E) TBAb K1-70 blocks M22-induced increases in cAMP. (FIG. 1F) HEK cells without TSHR treated with TSH. Each data point represents at least 3 replicates. Data shown are representative of 3 separate studies. In summary, these data show that the TBAb K1-70 blocks TSHR activation by TSH and the TSAb M22.

FIG. 2A to 2D shows that K1-70 AAV vector is able to express antibodies that bind TSHR. (FIG. 2A) Cell lysate (25 ug) was used to immunostain HEK cells that express the human TSHR). This labeling (green) of TSHR expressing cells is specific, as labeling is not observed in no primary antibody controls (FIG. 2B) or in the parent cell line that does not express TSHRs (FIG. 2C). K1-70 cell lysate has a similar staining pattern as the commercially available K1-70 antibody (FIG. 2D), which labels the TSHR best seen at cell-cell junctions. Scale bar=200 uM. Cells are counter-stained with DAPI to label nuclei.

FIG. 3 shows that AAV8-K1-70 vector produces an antibody that inhibits TSH action. Two concentrations of cell lysate collected from HEK cells transfected with the AAV8-K1-70 vector were tested for the ability to inhibit TSH action at a concentration of 150 ng/ml. The higher concentration of cell lysate protein (82.5 μg per 250 μl) blocked TSH activation of adenyl cyclase accumulation. N=3. P≤0.05. Lysates of cells that did not contain the vector did not inhibit TSH action at any concentration.

### DETAILED DESCRIPTION

Thyroid-related disease is associated with either inadequate production (i.e., hypothyroidism) or overproduction (i.e., hyperthyroidism) of thyroid hormones. Both types of disease are relatively common afflictions of man and animals. Hyperthyroidism, in particular, results from heightened secretion and release of thyroid hormones. In most species, this condition is less common than hypothyroidism. In humans the most common form of hyperthyroidism is Graves' disease, an immune disease in which autoantibodies bind to and activate the thyroid-stimulating hormone receptor (TSHR) leading to continual stimulation of thyroid hormone synthesis. These types of autoantibodies are also referred to as thyroid-stimulating antibodies (TSAbs).

Common signs of hyperthyroidism include nervousness, insomnia, high heart rate, eye disease and anxiety. Graves' disease is commonly treated with anti-thyroid drugs (e.g., propylthiouracil and methimazole), which suppress synthesis of thyroid hormones primarily by interfering with iodination of thyroglobulin by thyroid peroxidase. In addition, thyroid-blocking antibodies (TBAbs) have been identified which block TSHR stimulation by thyroid-stimulating hormone (TSH) or autoantibodies thereby reducing the overall synthesis of thyroid hormone (9, 10). For example, the thyroid-blocking monoclonal antibody K1-70 is under investigation as a treatment for Graves' disease (50).

However, infusion of antibodies, antibody fragments, or peptides that potentially block TSAbs, such as K1-70, last only for a short period of time and require repeated administration (6, 7). Improved methods of delivering clinically significant levels of thyroid-blocking antibodies which would not require repeated administrations would represent a significant advancement in the art for treating thyroid disorders, such as Graves' disease, Graves' orbitopathy, and thyroid cancer.

The present disclosure is based, at least in part, on the realization that blocking anti-TSHR antibodies may be delivered in a continuous manner using rAAV, e.g., rAAV8, to effectively block the stimulating effects of TSAbs or TSH on TSHR, thereby blocking or reducing the synthesis of thyroid hormone. By blocking or reducing the synthesis of thyroid hormone, the presently described methods and compositions for rAAV-based delivery of blocking anti-TSHR antibodies (e.g., K1-70) may be used to treat thyroid diseases, including Graves' disease, Graves' orbitopathy, and thyroid cancer without the requirement of repeated administrations.

### Definitions

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the present disclosure may be more readily understood, select terms are defined below.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “blocking anti-TSHR antibody” refers to an antibody (e.g., monoclonal antibody, polyclonal antibody, bispecific antibody, antibody fragment) which blocks or reduces the stimulating effect of thyroid-stimulating hormone (TSH) or a thyroid-stimulating autoantibody (“TSAb”) on the thyroid-stimulating hormone receptor (TSHR), thereby blocking or reducing the associated production of thyroid. Without being bound by theory, a blocking anti-TSHR antibody blocks directly or indirectly the binding of TSH or a TSAb to TSHR, thereby blocking the thyroid-stimulating effects of TSH and TSAb on TSHR.

The terms “biological activity” or “activity” of a protein, as used herein, refers to all inherent biological properties of the protein.

The terms “specific binding” or “specifically binding”, as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

An “antibody” (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The term “antigen-binding fragment” of an antibody (or simply “antibody fragment”), as used herein, refers to one or more fragments of an antibody that retain the ability to specifically bind to an antigen (e.g., TSHR). It has been shown that the antigen-binding function of an antibody can be performed by fragments of a full-length antibody. Such antibody embodiments may also be bispecific, dual specific, or multi-specific formats; specifically binding to two or more different antigens. Multispecific, dual specific, and bispecific antibody constructs are well known in the art and described and characterized in Kontermann (ed.), Bispecific Antibodies, Springer, N.Y. (2011), and Spiess et al., Mol. Immunol. 67(2):96-106 (2015).

Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more antigen binding portions of the disclosure linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences and their functional variations are known in the art.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds TSHR is substantially free of antibodies that specifically bind antigens other than TSHR). Moreover, an isolated antibody may be substantially free of other cellular material and/or chemicals.

The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human TSHR which can be generated using techniques well known in the art, such as, but not limited to, using human Ig phage libraries such as those disclosed in Jermutus et al., PCT publication No. WO 2005/007699 A2.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs (e.g., CDR3) has been replaced with murine CDR sequences.

The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which murine CDR sequences are introduced into human VH and VL sequences to replace the corresponding human CDR sequences. In one embodiment, humanized blocking anti-TSHR antibodies and antigen binding portions are provided. Such antibodies may be generated by obtaining murine blocking anti-TSHR antibodies using traditional hybridoma technology followed by humanization using in vitro genetic engineering, such as those disclosed in Kasaian et al PCT publication No. WO 2005/123126 A2.

In some embodiments, an antibody or a variant, derivative, analog or fragment thereof which immunospecifically binds to an antigen of interest and which comprises a framework (FR) region having substantially the amino acid sequence of a human antibody and a complementary determining region (CDR) has substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the framework regions are those of a human immunoglobulin consensus sequence. In one embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In another embodiment, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG 1, IgG2, IgG3 and IgG4. The humanized antibody may comprise sequences from more than one class or isotype, and particular constant domains may be selected to optimize desired effector functions using techniques well-known in the art.

In one embodiment, the framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In another embodiment, such mutations, however, will not be extensive. Usually, at least 80%, 85%, 90%, and or 95% of the humanized antibody residues will correspond to those of the parental FR and CDR sequences.

As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. In another embodiment, if two amino acids occur equally frequently, either can be included in the consensus sequence.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In an embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art. In one embodiment, the acceptor sequences known in the art may be used in the antibodies disclosed herein.

The term “thyroid-stimulating hormone receptor” or TSHR refers to the receptor for thyroid-stimulating hormone (TSH), a 27 kDa glycoprotein hormone produced by the anterior pituitary gland that comprises two dissimilar subunits. TSHR responds to thyroid-stimulating hormone (also known as “thyrotropin”) and stimulates the production of thyroxine (T4) and triiodothyronine (T3). The TSH receptor is a member of the G protein-coupled receptor superfamily of integral membrane proteins and is coupled to the Gs protein. It is primarily found on the surface of the thyroid epithelial cells, but also found on adipose tissue and fibroblasts. Upon binding circulating TSH, a G-protein signal cascade activates adenylyl cyclase and intracellular levels of cAMP rise. cAMP activates all functional aspects of the thyroid cell, including iodine pumping, thyroglobulin synthesis, iodination, endocytosis, proteolysis, thyroid peroxidase activity, and hormone release. Aliases include “TSHR”, “CHNG1”, “LGR3”, “HTSHR-I,” and thyrotropin receptor.

The receptor protein (764 amino acids—Accession No. NP_000360—SEQ ID ON: 5) represents a classical 7 membrane spanning, rhodopsin-like G protein coupled protein. Its structure has been solved with crystallization studies by the laboratory group of Reese-Smith (Sanders P, Young S, Sanders J, et al. Crystal structure of the TSH receptor (TSHR) bound to a blocking-type TSHR autoantibody. J Mol Endocrinol. 2011; 46:81-99, incorporated herein by reference). TSHR is a family member of cell surface receptors that includes luteinizing hormone (LH) and follicle stimulating hormone (FSH). It comprises a multimeric structure with the ligand-binding site located in the amino-terminus. One gene encodes the receptor which is translated into a single peptide undergoing cleavage into constituent subunits connected by a disulfide bond. The extracellular TSHR domain is cleaved by a cell surface metalloproteinase. This cleaved fragment is particularly immunogenic and its characteristics are likely to proximally underlie generation of TSI. The multimeric structure of the TSHR drives affinity maturation of the pathogenic autoantibodies in GD. Rearrangement of the interface between the extracellular domain-extracellular loop 1 appears to be critical to ligand-dependent receptor activation. TSIs mimic the actions of TSH and in so doing “fool” the TSHR into initiating signaling the epithelial cell to generate excessive amounts of thyroid hormones that in turn cause thyrotoxicosis through their exaggerated actions on target tissues. In addition to anti-TSHR antibodies that stimulate the receptor, others either block receptor activation or are neutral and assays are being developed to determine their levels in serum. It is important to stress that the same individual with GD can produce simultaneously both stimulatory and blocking antibodies, accounting perhaps for the rapid transition from hyperthyroidism to hypothyroidism observed in some patients. While the critical epitopes for ligand recognition and receptor activation have been identified, certain aspects of the molecular interactions between TSH and TSHR remain to be clarified.

An exemplary TSHR is Accession No. NP_000360 (754 amino acids) is human thyrotropin receptor isoform 1 precursor having the sequence:

| (SEQ ID NO: 5) | |
|---|---|
| 001 | MRPADLLQLV LLLDLPRDLG GMGCSSPPCE CHQEEDFRVT CKDIQRIPSL PPSTQTLKLI |
| 061 | ETHLRTIPSH AFSNLPNISR IYVSIDVTLQ QLESHSFYNL SKVTHIEIRN TRNLTYIDPD |
| 121 | ALKELPLLKF LGIFNTGLKM FPDLTKVYST DIFFILEITD NPYMTSIPVN AFQGLCNETL |
| 181 | TLKLYNNGFT SVQGYAFNGT KLDAVYLNKN KYLTVIDKDA FGGVYSGPSL LDVSQTSVTA |
| 241 | LPSKGLEHLK ELIARNTWTL KKLPLSLSFL HLTRADLSYP SHCCAFKNQK KIRGILESLM |
| 301 | CNESSMQSLR QRKSVNALNS PLHQEYEENL GDSIVGYKEK SKFQDTHNNA HYYVFFEEQE |
| 361 | DEIIGFGQEL KNPQEETLQA FDSHYDYTIC GDSEDMVCTP KSDEFNPCED IMGYKFLRIV |
| 421 | VWFVSLLALL GNVFVLLILL TSHYKLNVPR FLMCNLAFAD FCMGMYLLLI ASVDLYTHSE |
| 481 | YYNHAIDWQT GPGCNTAGFF TVFASELSVY TLTVITLERW YAITFAMRLD RKIRLRHACA |
| 541 | IMVGGWVCCF LLALLPLVGI SSYAKVSICL PMDTETPLAL AYIVFVLTLN IVAFVIVCCC |
| 601 | YVKIYITVRN PQYNPGDKDT KIAKRMAVLI FTDFICMAPI SFYALSAILN KPLITVSNSK |
| 661 | ILLVLFYPLN SCANPFLYAI FTKAFQRDVF ILLSKFGICK RQAQAYRGQR VPPKNSTDIQ |
| 721 | VQKVTHEMRQ GLHNMEDVYE LIENSHLTPK KQGQISEEYM QTVL. |

The term “activity” includes activities such as the binding specificity/affinity of an antibody for an antigen.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “polynucleotide” as referred to herein means a polymeric form of two or more nucleotides, either ribonucleotides or deoxynucleotides or a modified form of either type of nucleotide. The term includes single and double stranded forms of DNA, and can encompass the genome or a recombinant genome of an AAV.

The term “isolated polynucleotide” as used herein shall mean a polynucleotide (e.g., of genomic, cDNA, or synthetic origin, or some combination thereof) that, by virtue of its origin, is not associated with all or a portion of a polynucleotide with which the “isolated polynucleotide” is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses (e.g., AAV8)), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences. Protein constructs of the present disclosure may be expressed, and purified using expression vectors and host cells known in the art, including expression cassettes, vectors, recombinant host cells and methods for the recombinant expression and proteolytic processing of recombinant polyproteins and pre-proteins from a single open reading frame (e.g., WO 2007/014162 incorporated herein by reference).

The term “agonist”, as used herein, refers to a modulator that, when contacted with a molecule of interest, causes an increase in the magnitude of a certain activity or function of the molecule compared to the magnitude of the activity or function observed in the absence of the agonist. A TSAb is an example of an agonist because it is an anti-TSHR antibody which stimulates the synthesis of thyroid hormone in a manner similar to thyroid-stimulating hormone.

The term “antagonist” or “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of TSHR, e.g., by blocking access to TSH or TSAbs and thereby blocking or reducing the effect of TSH and/or TSAbs on TSHR, and the concomitant reduction in the synthesis of thyroid hormone.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). As one example, the disorder is Graves' disease.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

### Blocking Anti-TSHR Antibodies

The present methods and compositions contemplate the use of any known or available blocking anti-TSHR antibodies, or fragments thereof.

Blocking anti-TSHR antibodies that may be used in the instant methods and compositions can include those disclosed in U.S. Pat. Nos. 9,040,670, 8,029,790, 8,501,415, and 8,840,891, each of which are disclosed herein by reference in their entireties.

Blocking anti-TSHR antibodies that may be used in the instant methods and compositions can also include those disclosed in (i) Furmaniak et al., “Blocking type TSH receptor antibodies,” Auto Immun Highlights, 2013, 4(1): 11-26, (ii) Sanders et al., “TSH receptor monoclonal antibodies with agonist, antagonist, and inverse agonist activities,” Methods Enzymol, 2010, 485: 383-420, (iii) Terry J. Smith, “TSHR as a therapeutic target in Graves” disease,” Exper Opin Ther Targets, 2017, 21(4): 427-432, (iv) Jeffreys et al., “Characterization of the thyrotropin binding pocket,” Thyroid, 2002 December; 12(12):1051-61, (iv) Smith et al., “TSH receptor—autoantibody interactions,” Horn Metab Res. 2009 June; 41(6):448-55, (v) Sanders et al., “A human monoclonal autoantibody to the thyrotropin receptor with thyroid-stimulating blocking activity,” Thyroid, 2008 July; 18(7):735-46, (vi) Evans et al., “Monoclonal autoantibodies to the TSH receptor, one with stimulating activity and one with blocking activity, obtained from the same blood sample,” Clin Endocrinol (Oxf)., 2010, September; 73(3):404-12, and (vii) Bryant et al., “Identification of thyroid blocking antibodies and receptor epitopes in autoimmune hypothyroidism by affinity purification using synthetic TSH receptor peptides,” Autoimmunity, 22, pp. 69-79 (1995), each of which are disclosed herein by reference in their entireties.

In certain embodiments, the blocking anti-TSHR antibody is K1-70 comprising: (i) a heavy chain sequence of SEQ ID NO: 1 (Accession No. 2XWT_A, “Chain A, THYROID BLOCKING HUMAN AUTOANTIBODY K1-70 HEAVY CHAIN”):

(SEQ ID NO: 1)
EVQLVQSGAE VKKPGQSLKI SCKASGYSLT DNWIGWVRQK
PGKGLEWMGI IYPGDSDTRY SPSFQGQVTI SADKSINTAY
LQWSSLKASD TAIYYCVGLD WNYNPLRYWG PGTLVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKKVEPK S,

and (ii) a light chain sequence of SEQ ID NO: 2 (Accession No. 2XWT_B, “Chain B, THYROID BLOCKING HUMAN AUTOANTIBODY K1-70 LIGHT CHAIN”):

(SEQ ID NO: 2)
QSVLTQPPSV SAAPGQKVTI SCSGSSSDIG SNYVSWYQQF
PGTAPKLLIY DNNKRPSAIP DRFSGSKSGT SATLGITGLQ
TGDEADYYCG TWDSRLGIAV FGGGTQLTVL GQPKAAPSVT
LFPPSSEELQ ANKATLVCLV SDFYPGAVTV AWKADGSPVK
VGVETTKPSK QSNNKYAASS YLSLTPEQWK SHRSYSCRVT
HEGSTVEKTV APTE.

See Sanders, J. et al., “Human monoclonal thyroid stimulating autoantibody,” Lancet 362 (9378), 126-128 (2003) (which is incorporated herein by reference).

In certain other embodiments, the blocking anti-TSHR antibody is mouse monoclonal antibody, CS-17 comprising: (i) a light chain sequence of SEQ ID NO: 3 (Accession No. AYH92450, “CS-17 TSH receptor monoclonal antibody light chain variable region”):

(SEQ ID NO: 3)
ELVFTQPPAI MSASPGEKVT ISCSASSSVS YMCWFQQKPG
SSPKPWIYRT SNLASGVPAR FSGSGSGTSY SLTISSMEAE
DAATYYCQQY HSYPLTFGAG TKLELKRADA APTVSIFP,

and (ii) a light chain sequence of SEQ ID NO: 4 (Accession No. AYH92449, “CS-17 TSH receptor monoclonal antibody heavy chain variable region, partial [Mus musculus]”):

(SEQ ID NO: 4)
EVQLLESGPE LVKPGASVKM SCKASGYTFT SYIIHWVKQK
PGQGLEWIGY INLYNDGTNY NEKFTGKATL TSDKSSSTAY
MELSSLTSED SAVYYCARED YYGRVADFDV WGAGTTVTVS
SAKTT.

In certain embodiments, the blocking anti-TSHR antibodies target to or bind several regions of the TSHR receptor, including those comprising amino acids 32-41,36-42, 246-260, 277-296, and 381-385 of SEQ ID NO: 5 (TSHR) and which can block TSH binding in the ligand-binding pocket. A reference sequence of TSHR is Accession No. NP_000360 (754 amino acids) which is a human thyrotropin receptor isoform 1 precursor having the sequence:

| (SEQ ID NO: 5) | |
|---|---|
| 001 | MRPADLLQLV LLLDLPRDLG GMGCSSPPCE CHQEEDFRVT CKDIQRIPSL PPSTQTLKLI |
| 061 | ETHLRTIPSH AFSNLPNISR IYVSIDVTLQ QLESHSFYNL SKVTHIEIRN TRNLTYIDPD |
| 121 | ALKELPLLKF LGIFNTGLKM FPDLTKVYST DIFFILEITD NPYMTSIPVN AFQGLCNETL |
| 181 | TLKLYNNGFT SVQGYAFNGT KLDAVYLNKN KYLTVIDKDA FGGVYSGPSL LDVSQTSVTA |
| 241 | LPSKGLEHLK ELIARNTWTL KKLPLSLSFL HLTRADLSYP SHCCAFKNQK KIRGILESLM |
| 301 | CNESSMQSLR QRKSVNALNS PLHQEYEENL GDSIVGYKEK SKFQDTHNNA HYYVFFEEQE |
| 361 | DEIIGFGQEL KNPQEETLQA FDSHYDYTIC GDSEDMVCTP KSDEFNPCED IMGYKFLRIV |
| 421 | VWFVSLLALL GNVFVLLILL TSHYKLNVPR FLMCNLAFAD FCMGMYLLLI ASVDLYTHSE |
| 481 | YYNHAIDWQT GPGCNTAGFF TVFASELSVY TLTVITLERW YAITFAMRLD RKIRLRHACA |
| 541 | IMVGGWVCCF LLALLPLVGI SSYAKVSICL PMDTETPLAL AYIVFVLTLN IVAFVIVCCC |
| 601 | YVKIYITVRN PQYNPGDKDT KIAKRMAVLI FTDFICMAPI SFYALSAILN KPLITVSNSK |
| 661 | ILLVLFYPLN SCANPFLYAI FTKAFQRDVF ILLSKFGICK RQAQAYRGQR VPPKNSTDIQ |
| 721 | VQKVTHEMRQ GLHNMEDVYE LIENSHLTPK KQGQISEEYM QTVL. |

A blocking anti-TSHR antibody as described herein may be a protein (e.g., an antibody, or antigen-binding fragments thereof such as scFv-Fc, (Fab′)₂, minibody, Fab, diabody, scFv, or dAb), or a siRNA (e.g., siRNA specific for TSHR).

In some embodiments, an blocking anti-TSHR antibody is a blocking anti-TSHR antibody, or antibody fragment (e.g., scFv-Fc, (Fab′)₂, minibody, Fab, diabody, scFv, dAb, or tri-Ab). A blocking anti-TSHR antibody or antigen-binding fragments thereof binds to TSHR with a high specificity (i.e., its likelihood and ability to bind to TSHR is greater than its likelihood and ability to bind other proteins). Rodrigo et al. (Antibodies 2015, 4(3), 259-277), Chapter 12 of Therapeutic Antibody Engineering: Current and Future Advances Driving the Strongest Growth Area in the Pharmaceutical Industry (ISBN 9781907568374) provide a review of various types of antibody fragments, including single-chain antibodies, each reference being incorporated herein by reference in its entirety. In some embodiments, a blocking anti-TSHR antibody encoded by a nucleic acid in an rAAV particle (e.g., an rAAV serotype 8 particle) as disclosed herein comprises a heavy chain and a light chain.

In some embodiments, a blocking anti-TSHR antibody is a single-chain antibody. A single-chain antibody is an antibody that comprises at least one heavy chain or fragment of a heavy chain, and at least one light chain or fragment of a light chain, wherein the heavy chain or fragment of a heavy chain is connected to the light chain or fragment of a light chain. The advantage of a single-chain antibody for delivery via rAAV particles is that single-chain antibodies require expression of only a single gene, which is more easily included within the rAAV genome compared to multiple genes. A single-chain variable fragment (scFv) is an example of a single-chain antibody.

In some embodiments, a heavy chain or fragment of a heavy chain is connected to a light chain or fragment of a light chain by a linker. In some embodiments, a linker is a polypeptide.

Single domain blocking anti-TSHR antibodies are also contemplated herein.

A non-limiting example of a blocking anti-TSHR antibody is K1-70 comprising a heavy chain (SEQ ID NO: 1) and a light chain (SEQ ID NO: 2).

In some embodiments, a blocking anti-TSHR antibody or fragment thereof comprises a contiguous amino acid sequence (e.g., at least 10, 20, 30, 40, 50, 60, 70, 80, 90, 100, 150, 200, 250, 300, 350, 400, or 450 amino acids long) of SEQ ID NOs: 1-4 and a contiguous amino acid sequence (e.g., at least 10, 20, 30, 40, 50, 60, 70, 80, 90, 100, 150, or 200 amino acids long) of SEQ ID NOs: 1-4.

Any one of the blocking anti-TSHR antibodies described herein may have a heavy chain or fragment thereof comprising a contiguous amino acid sequence (e.g., at least 10, 20, 30, 40, 50, 60, 70, 80, 90, 100, 150, 200, 250, 300, 350, 400, or 450 amino acids long) of SEQ ID NO: 1. In some embodiments, a heavy chain fragment of a blocking anti-TSHR antibody or fragment thereof comprises a sequence that is at least 50% (e.g., at least 60, 70, 80, 90, 95, 97, 98, 99, 99.1, 99.2, 99.3, 994, 99.5, 99.6, 99.7, 99.8, or at least 99.9%) homologous to that of SEQ ID NO: 1.

Any one of the blocking anti-TSHR antibodies described herein may have a light chain or fragment thereof comprising a contiguous amino acid sequence (e.g., at least 10, 20, 30, 40, 50, 60, 70, 80, 90, 100, 150, or 200 amino acids long) of SEQ ID NO: 2. In some embodiments, a light chain fragment of an anti-VEGF antibody or fragment thereof comprises a sequence that is at least 50% (e.g., at least 60, 70, 80, 90, 95, 97, 98, 99, 99.1, 99.2, 99.3, 99.4, 99.5, 99.6, 99.7, 99.8, or at least 99.9%) homologous to that of SEQ ID NOs: 2.

Any one of the blocking anti-TSHR antibodies described herein may have a heavy chain or fragment thereof comprising a contiguous amino acid sequence (e.g., at least 10, 20, 30, 40, 50, 60, 70, 80, 90, 100, 150, 200, 250, 300, 350, 400, or 450 amino acids long) of SEQ ID NO: 4. In some embodiments, a heavy chain fragment of a blocking anti-TSHR antibody or fragment thereof comprises a sequence that is at least 50% (e.g., at least 60, 70, 80, 90, 95, 97, 98, 99, 99.1, 99.2, 99.3, 994, 99.5, 99.6, 99.7, 99.8, or at least 99.9%) homologous to that of SEQ ID NO: 4.

Any one of the blocking anti-TSHR antibodies described herein may have a light chain or fragment thereof comprising a contiguous amino acid sequence (e.g., at least 10, 20, 30, 40, 50, 60, 70, 80, 90, 100, 150, or 200 amino acids long) of SEQ ID NO: 3. In some embodiments, a light chain fragment of an anti-VEGF antibody or fragment thereof comprises a sequence that is at least 50% (e.g., at least 60, 70, 80, 90, 95, 97, 98, 99, 99.1, 99.2, 99.3, 99.4, 99.5, 99.6, 99.7, 99.8, or at least 99.9%) homologous to that of SEQ ID NO: 3.

In some embodiments, a blocking anti-TSHR antibody comprises two heavy chains and two light chains. In some embodiments, a blocking anti-TSHR antibody comprises a heavy chain linked to a light chain via a linker. In some embodiments, a blocking anti-TSHR antibody comprises a heavy chain fragment (e.g., Fv or Fab regions) linked to a light chain (e.g., Fv or Fab regions) via a linker.

A linker, as defined herein, is a polypeptide the primary function of which is to connect two regions of a blocking anti-TSHR antibody (e.g., a heavy chain fragment and a light chain fragment of a blocking anti-TSHR antibody). In some embodiments, a linker is flexible and relatively structure-less. In some embodiments, a linker is 5-100 amino acids long (e.g., 5-10, 5-20, 10-15, 10-20, 15-30, 20-30, 10-30, 5-30, 10-40, 20-40, 25-50, 20-50, 30-50, or 40-50 amino acids long). In some embodiments, a linker is at least 5 amino acids long (e.g., at least 5, at least 10, at least 15, at least 20, at least 25, at least 30, at least 40, at least 50, or at least 100 amino acids long). In some embodiments, a linker comprises mostly of glycines and serines. SEQ ID NO: 6 is an example of a linker sequence. SEQ ID NO: 7 is an example of a sequence of a polynucleotide that encodes the linker of SEQ ID NO: 6.

Example amino acid sequence of a linker:

(SEQ ID NO: 6)
GGSGGGSGGGGSGGGSGGGG.

Example nucleic acid sequence encoding a linker:

(SEQ ID NO: 7)
GGCGGCAGCGGCGGCGGCAGCGGCGGCGGCGGCAGCGGCGGCGGCAGCG
GCGGCGGCGGC.

### Secretion Signal

In some embodiments, any of the blocking anti-TSHR antibodies disclosed herein comprises a secretion signal. The secretion signal allows the blocking anti-TSHR antibodies to secrete outside of a cell that expresses it, where it can interact with TSHR and block the activation of TSHR by TSH and/or TSAbs, thereby shutting down the synthesis of thyroid hormone. A non-limiting example of a secretion signal is provided in SEQ ID NO: 8. SEQ ID NO: 9 provides an example of a polynucleotide encoding a secretion signal.

Example amino acid sequence of a secretion signal:

(SEQ ID NO: 8)
METDTLLLWVLLLWVPGSTGD.

Example of nucleic acid sequence of a polynucleotide encoding a secretion signal:

(SEQ ID NO: 9)
ATGGAGACCGACACCCTGCTGCTGTGGGTGCTGCTGCTGTGGGTGCCCG
GCAGCACCGGCGAC.

Other non-limiting examples of secretion signals are provided in Table 1. In some embodiments, a secretion signal in a blocking anti-TSHR antibody comprises any one of SEQ ID NOs: 10-21.

TABLE 1
Examples of secretion signals

| | | | SEQ |
|---|---|---|---|
| | UniProt | | ID |
| Protein | Number | Secretion Signal Sequence | NO. |
| CNTF | P26992 | MAAPVPWACCAVLAAAAAVVYA | 10 |
| PEDF | P36955 | MQALVLLLCIGALLGHSSC | 11 |
| FGF10 | O15520 | MWKWILTHCASAFPHLPGCCCCCFLLLFLV | 12 |
| | | SSVPVTC | |
| PDGF-A | P04085 | MRTLACLLLLGCGYLAHVLA | 13 |
| Gas6 | Q14393 | MAPSLSPGPAALRRAPQLLLLLLAAECALA | 14 |
| TIMP3 | P35625 | MTPWLGLIVLLGSWSLGDWGAEA | 15 |
| VEGF-A | P15692 | MNFLLSWVHWSLALLLYLHHAKWSQA | 16 |
| TGF-b 1 | P01137 | MPPSGLRLLLLLLPLLWLLVLTPGRPAAG | 17 |
| CFH | P08603 | MRLLAKIICLMLWAICVA | 18 |
| IL-8 | P10145 | MTSKLAVALLAAFLISAALC | 19 |
| MCP-1 | P13500 | MKVSAALLCLLLIAATFIPQGLA | 20 |
| GDNF | P39905 | MKLWDVVAVCLVLLHTASA | 21 |

### Antibody Modifications

Antibodies of the disclosure may be modified with a detectable label, including, but not limited to, an enzyme, prosthetic group, fluorescent material, luminescent material, bioluminescent material, radioactive material, positron emitting metal, nonradioactive paramagnetic metal ion, and affinity label for detection and isolation of the blocking antibodies. The detectable substance may be coupled or conjugated either directly to the polypeptides of the disclosure or indirectly, through an intermediate (such as, for example, a linker) using suitable techniques. Non-limiting examples of suitable enzymes include horseradish peroxidase, alkaline phosphatase, β-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (131I, 125I, 123I, 121I), carbon (14C), sulfur (35S), tritium (3H), indium (115mIn, 113mIn, 112In, 111In), and technetium (99Tc, 99mTc), thallium (201Ti), gallium (68Ga, 67Ga), palladium (103Pd), molybdenum (99Mo), xenon (133Xe), fluorine (18F), 153Sm, Lu, 159Gd, 149Pm, 140La, 175Yb, 166Ho, 90Y, 47Sc, 86R, 188Re, 142Pr, 105Rh, 97Ru, 68Ge, 57Co, 65Zn, 85Sr, 32P, 153Gd, 169Yb, 51Cr, 54Mn, 75Se, and tin (113Sn, 117Sn). The detectable substance may be coupled or conjugated either directly to the blocking antibodies of the disclosure or indirectly, through an intermediate (such as, for example, a linker) using suitable techniques.

In some embodiments, it is useful to know if the gene product that intended to be delivered by an rAAV particle is actually delivered, and if delivered, whether the gene product is being expressed. One way to validate delivery and expression of a blocking anti-TSHR antibody is by designing a detectable molecule to be expressed when the blocking anti-TSHR antibody is expressed. In some embodiments, a detectable molecule is fused to the blocking anti-TSHR antibody. In embodiments, a detectable molecule is expressed when the blocking anti-TSHR antibody is expressed, but becomes cleaved away from the blocking anti-TSHR antibody by the use of a self-cleaving peptide connecting the detectable molecule and the blocking anti-TSHR antibody. In some embodiments, the self-cleaving peptide is a P2A peptide. In some embodiments, a P2A peptide comprises a sequence of SEQ ID NO: 22. In some embodiments, a P2A peptide has a sequence of SEQ ID NO: 23.

Example of amino acid sequence of a self-cleaving peptide:

(SEQ ID NO: 22)
AAAATNFSLLKQAGDVEENPGP.

Example of a nucleic acid sequence encoding a self-cleaving peptide:

(SEQ ID NO: 23)
GCCGCCGCCGCCACCAACTTCAGCCTGCTGAAGCAGGCCGGCGACGTGG
AGGAGAACCCCGGCCCC.

In some embodiments, a detectable molecule is a fluorescent protein, a bioluminescent protein, or a protein that provides color (e.g., β-galactosidase, β-lactamases, β-glucuronidase and spheriodenone). In some embodiments, a detectable molecule is a fluorescent, bioluminescent or enzymatic protein or functional peptide or functional polypeptide thereof.

In some embodiments, fluorescent protein is a blue fluorescent protein, a cyan fluorescent protein, a green fluorescent protein, a yellow fluorescent protein, an orange fluorescent protein, a red fluorescent protein, or functional peptides or polypeptides thereof. A blue fluorescent protein may be azurite, EBFP, EBFP2, mTagBFP, or Y66H. A cyan fluorescent protein may be ECFP, AmCyanl, Cerulean, CyPet, mECFP, Midori-ishi Cyan, mTFP1, or TagCFP. A Green fluorescent protein may be AcGFP, Azami Green, EGFP, Emarald, GFP or a mutated form of GFP (e.g., GFP-S65T, mWasabi, Stemmer, Superfolder GFP, TagGFP, TurboGFP, and ZsGreen). A yellow fluorescent protein may be EYFP, mBanana, mCitrine, PhiYFp, TagYFP, Topaz, Venus, YPet, or ZsYellowl. An orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP, or TagRFP-T. A red fluorescent protein may be AQ142, AsRed2, dKeima-Tandem, HcRedl, tHcRed, Jred, mApple, mCherry, mPlum, mRasberry, mRFP1, mRuby or mStrawberry.

SEQ ID NOs: 24 and 25 provide non-limiting examples of amino acid and nucleic acid sequences for GFP.

Example of amino acid sequence of GFP:

(SEQ ID NO: 24)
MSKGEELFTGVVPILVELDGDVNGHKFSVSGEGEGDATYGKVNGHKFSV
SGEGEGDATYGKTTLTYGVQCFSRYPDHMKQHDFFKSAMPEGYVQERTI
FFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLP
DNHYLSTQSALSKDPNEKRDHMVLLEFVTAAGITHGMDELY.

Example of a nucleic acid sequence encoding GFP:

(SEQ ID NO: 25)
ATGAGCAAGGGCGAGGAGCTGTTCACCGGCGTGGTGCCCATCCTGGTGG
AGCTGGACGGCGACGTGAACGGCCACAAGTTCAGCGTGAGCGGCGAGGG
CGAGGGCGACGCCACCTACGGCAAGCTGACCCTGAAGTTCATCTGCACC
ACCGGCAAGCTGCCCGTGCCCTGGCCCACCCTGGTGACCACCCTGACCT
ACGGCGTGCAGTGCTTCAGCCGCTACCCCGACCACATGAAGCAGCACGA
CTTCTTCAAGAGCGCCATGCCCGAGGGCTACGTGCAGGAGCGCACCATC
TTCTTCAAGGACGACGGCAACTACAAGACCCGCGCCGAGGTGAAGTTCG
AGGGCGACACCCTGGTGAACCGCATCGAGCTGAAGGGCATCGACTTCAA
GGAGGACGGCAACATCCTGGGCCACAAGCTGCGCATCGAGCTGAAGGGC
ATCGACTTCAAGGAGGACGGCAACATCCTGGGCCACAAGCTGAAGGTGA
ACTTCAAGATCCGCCACAACATCGAGGACGGCAGCGTGCAGCTGGCCGA
CCACTACCAGCAGAACACCCCCATCGGCGACGGCCCCGTGCTGCTGCCC
GACAACCACTACCTGAGCACCCAGAGCGCCCTGAGCAAGGACCCCAACG
AGAAGCGCGACCACATGGTGCTGCTGGAGTTCGTGACCGCCGCCGGCAT
CACCCACGGCATGGACGAGCTGTACAAGTAA.

In some embodiments, a detectable molecule is a bioluminescent protein, or functional peptide or polypeptide thereof. Non-limiting examples of bioluminescent proteins are firefly luciferase, click-beetle luciferase, Renilla luciferase, or luciferase from Oplophorus gracilirostris.

In some embodiments, a detectable molecule may be any polypeptide or protein that can be detected using methods known in the art. Non-limiting methods of detection are fluorescence imaging, luminescent imaging, bright filed imaging.

In some embodiments, any of the blocking anti-TSHR antibodies provided herein comprise a polyhistidine tag. In some embodiments, the polyhistidine tag comprises two, three, four, five six, seven, eight, nine, ten, or more consecutive histidine residues. In some embodiments, the polyhistidine tag is comprised at the N-terminus of any of the blocking anti-TSHR antibodies provided herein. In some embodiments, the polyhistidine tag is comprised at the C-terminus of any of the blocking anti-TSHR antibodies provided herein. In some embodiments, the polyhistidine tag is comprised within any of the blocking anti-TSHR antibodies provided herein. In some embodiments, the polyhistidine tag is fused directly to any of the blocking anti-TSHR antibodies provided herein. In some embodiments, the polyhistidine tag is fused to any of the blocking anti-TSHR antibodies provided herein via a linker.

While polyhistidine is a common and useful purification tag, it is well known in the art that other expressable peptide sequences can act as tags for both purification and localization of the antibody in later pre-clinical studies. In many instances, use of protein A affinity is used to purify antibodies.

### Production of Blocking Anti-TSHR Antibodies

Numerous methods may be used for obtaining antibodies, or antigen binding fragments thereof, of the disclosure. For example, antibodies can be produced using recombinant DNA methods. Monoclonal antibodies may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET or BIACORE) analysis, to identify one or more hybridomas that produce an antibody that specifically binds to a specified antigen. Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597WO92/18619; WO 91/17271; WO 92/20791; WO 92/15679; WO 93/01288; WO 92/01047; WO 92/09690; and WO 90/02809, each of which are incorproated herein by reference.

In addition to the use of display libraries, the specified antigen (e.g., TSHR) can be used to immunize a non-human animal, e.g., a rodent, e.g., a mouse, hamster, or rat. In one embodiment, the non-human animal is a mouse.

In another embodiment, a monoclonal antibody is obtained from the non-human animal, and then modified, e.g., chimeric, using suitable recombinant DNA techniques. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B, each of which are incorporated by reference.

For additional antibody production techniques, see Antibodies: A Laboratory Manual, eds. Harlow et al., Cold Spring Harbor Laboratory, 1988. The present disclosure is not necessarily limited to any particular source, method of production, or other special characteristics of an antibody.

Some aspects of the present disclosure relate to host cells transformed with a polynucleotide or vector. Host cells may be a prokaryotic or eukaryotic cell. The polynucleotide or vector which is present in the host cell may either be integrated into the genome of the host cell or it may be maintained extra-chromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies, antigen binding fragments or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments. Furthermore, transgenic animals, preferably mammals, comprising the aforementioned host cells may be used for the large-scale production of the antibody or antibody fragments.

The transformed host cells can be grown in fermenters and cultured using any suitable techniques to achieve optimal cell growth. Once expressed, the whole antibodies, their dimers, individual light and heavy chains, other immunoglobulin forms, or antigen binding fragments, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen binding fragments can then be isolated from the growth medium, cellular lysates, or cellular membrane fractions. The isolation and purification of the, e.g., microbially expressed antibodies or antigen binding fragments may be by any conventional means such as, for example, preparative chromatographic separations and immunological separations such as those involving the use of monoclonal or polyclonal antibodies directed, e.g., against the constant region of the antibody.

Aspects of the disclosure relate to a hybridoma, which provides an indefinitely prolonged source of monoclonal antibodies. As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. In some embodiments, heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment may be undertaken by culturing a modified recombinant host under culture conditions appropriate for the growth of the host cells and the expression of the coding sequences. The antibodies or antigen binding fragments may be recovered by isolating them from the culture. The expression systems may be designed to include signal peptides so that the resulting antibodies are secreted into the medium; however, intracellular production is also possible.

The disclosure also includes a polynucleotide encoding at least a variable region of an immunoglobulin chain of any of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer or a globin intron in mammalian and other animal cells.

In some embodiments, the herein described rAAV comprises one or more regions comprising a sequence that facilitates expression of the nucleic acid (e.g., the heterologous nucleic acid, e.g., the blocking anti-TSHR antibody), e.g., expression control sequences operatively linked to the nucleic acid. The promoter may be, for example, a constitutive promoter, tissue-specific promoter, inducible promoter, or a synthetic promoter. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter (e.g. chicken β-actin promoter) and human elongation factor-1 α (EF-1α) promoter. In some embodiments, a promoter is a truncated chimeric CMV-chicken β-actin (smCBA) promoter. SEQ ID NO: 26 provides a non-limiting example of an smCBA promoter:

Example nucleic acid sequence of a smCBA promoter:

(SEQ ID NO: 26)
GAATTCGGTACCCTAGTTATTAATAGTAATCAATTACGGGGTCATTAGT
TCATAGCCCATATATGGAGTTCCGCGTTACATAACTTACGGTAAATGGC
CCGCCTGGCTGACCGCCCAACGACCCCCGCCCATTGACGTCAATAATGA
CGTATGTTCCCATAGTAACGCCAATAGGGACTTTCCATTGACGTCAATG
GGTGGACTATTTACGGTAAACTGCCCACTTGGCAGTACATCAAGTGTAT
CATATGCCAAGTACGCCCCCTATTGACGTCAATGACGGTAAATGGCCCG
CCTGGCATTATGCCCAGTACATGACCTTATGGGACTTTCCTACTTGGCA
GTACATCTACGTATTAGTCATCGCTATTACCATGGTCGAGGTGAGCCCC
ACGTTCTGCTTCACTCTCCCCATCTCCCCCCCCTCCCCACCCCCAATTT
TGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGC
GGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAA
GCGAAGCGCGCGGCGGGCGGGAGTCGCTGCGACGCTGCCTTCGCCCCGT
GCCCCGCTCCGCCGCCGCCTCGCGCCGCCCGCCCCGGCTCTGACTGACC
GCGTTACTCCCACAGGTGAGCGGGCGGGACGGCCCTTCTCCTCCGGGCT
GTAATTAGCGCTTGGTTTAATGACGGCTTGTTTCTTTTCTGTGGCTGCG
TGAAAGCCTTGAGGGGCTCCGGGAGCTAGAGCCTCTGCTAACCATGTTC
ATGCCTTCTTCTTTTTCCTACAGCTCCTGGGCAACGTGCTGGTTATTGT
GCTGTCTCATCATTTTGGCAAAGAATTC.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. Non-limiting examples of such promoters that may be used include airway epithelial cell-specific promoters.

Synthetic promoters are also contemplated herein. A synthetic promoter may comprise, for example, regions of known promoters, regulatory elements, transcription factor binding sites, enhancer elements, repressor elements, and the like.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and have been described previously. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains or only one. Likewise, polynucleotides may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen binding fragment; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen binding fragment). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

### Recombinant AAV Particles

An rAAV particle as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). In a preferred embodiment, the serotype is 8, i.e., AAV8. Genbank reference numbers for sequences of AAV serotypes 1, 2, 3, 3B, 4, 5, 6, 7, 8, 9, 10, 11, 12, and 13 are listed in patent publication WO 2012/064960, which is incorporated herein by reference in its entirety. An rAAV particle may be an empty AAV capsid, or may comprise an AAV capsid further comprises a genetic load (i.e., a recombinant nucleic acid vector that expresses a gene of interest, such as a blocking anti-TSHR antibody that is carried by the rAAV particle into a cell) that is to be delivered to a cell. An rAAV particle may be chimeric.

Pseudotyping refers to using the capsid of one serotype and the genome of another serotype, or the mixing of a capsid and genome from different viral serotypes. These serotypes are denoted using a slash, so that AAV2/5 indicates a virus containing the genome of serotype 2 packaged in the capsid from serotype 5.

As used herein, the serotype of an rAAV viral particle refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV particles may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

In some embodiments, a gene of interest encodes a therapeutic protein (e.g., a blocking anti-TSHR antibody) or therapeutic RNA. In some embodiments, a therapeutic gene encodes an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, an anticoagulant, a nuclease, guide RNA or other nucleic acid or protein for gene editing.

Any one of the rAAV particles provided herein may have capsid proteins that have amino acids of different serotypes outside of the VP1 region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

### Compositions and Formulations

In some embodiments, the composition comprises a pharmaceutically acceptable carrier. The term “carrier” refers to a diluent, adjuvant, excipient, or vehicle with which the rAAV particle is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

Typically, such compositions may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms of the rAAV particle compositions suitable for injectable use include sterile aqueous solutions or dispersions. In some embodiments, the form is a sterile fluid that can be delivered by syringe. In some embodiments, the form is stable under the conditions of manufacture and storage and is preserved against the contaminating action of microorganisms, such as bacteria and fungi. In some embodiments, the form is sterile. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subretinal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

### Kits and Cells

Aspects of the disclosure relate to kits. In some embodiments, a kit comprising any one of the compositions disclosed herein comprising any one of the rAAV particles disclosed herein, comprising a nucleic acid encoding any of the blocking anti-TSHR antibodies disclosed herein. In some embodiments, a kit comprises a dry composition and one or more solvents. In some embodiments, a kit comprises a dry composition, one or more solvents, and instructions for adding the solvent to the dry composition of rAAV particles. In some embodiments, a kit comprises multiple vial or containers comprising rAAV particle compositions for different administrations. In some embodiments, a kit comprises instructions for administering a constituted composition to a subject.

Some embodiments of this disclosure provide cells comprising any of the anti-TSHR antibodies, nucleic acid molecules encoding same, or rAAV encoding same, as provided herein. In some embodiments, the cells comprise nucleotide constructs that encode any of the compositions of matter provided herein. In some embodiments, the cells comprise any of the nucleotides or vectors provided herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences (e.g., anti-TSHR antibodies).

### Delivery Methods

Provided herein is a method of delivering blocking anti-TSHR antibodies to a subject in need (e.g., a subject with Graves' disease). In some embodiments, the blocking anti-TSHR antibodies are delivered to the subject by administering any one of the compositions disclosed herein to a subject. In some embodiments, “administering” or “administration” means providing a material to a subject in a manner that is pharmacologically useful. In some embodiments, a composition is applied topically. In some embodiments, a composition is administered via peritnoneal injection.

In some embodiments, a rAAV particle or the herein compositions are administered to a subject enterally. In some embodiments, a rAAV particle or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

The AAV particles or polynucleotides may be delivered in the form of a composition, such as a composition comprising the active ingredient, such as AAV particles described herein, and a pharmaceutically acceptable carrier as described herein. The AAV particles or polynucleotides may be prepared in a variety of compositions, and may also be formulated in appropriate pharmaceutical vehicles for administration to human or animal subjects. In some embodiments, where first and second AAV particles are utilized, the first and second AAV particles may be contained within the same composition or within different compositions and may be administered together or separately.

In some embodiments, the AAV particles administered to a subject may be provided in a composition having a concentration on the order ranging from 10¹ to 10¹⁵ particles/ml or 10³ to 10¹⁰ particles/ml, or any values there between for either range, such as for example, about 10¹, 10², 10³, 10⁴, 10⁵, 10⁶, 10⁷, 10⁸, 10⁹, 10¹⁰, 10¹¹, 10¹², 10¹³, 10¹⁴, or 10¹⁵ or more particles/ml. In one embodiment, AAV particles of higher than 10¹³ particles/ml are be administered. In some embodiments, the number of AAV particles administered to a subject may be on the order ranging from 10⁶ to 10¹⁴ vector genomes (vgs)/ml or 10³ to 10¹⁵ vgs/ml, or any values therebetween for either range, such as for example, about 10⁶, 10⁷, 10⁸, 10⁹, 10¹⁰, 10¹¹, 10¹², 10¹³, or 10¹⁴ vgs/ml. In one embodiment, AAV particles of higher than 10¹³ vgs/ml are be administered. The AAV particles may be administered as a single dose, or divided into two or more administrations as may be required to achieve therapy of the particular disease or disorder being treated. In some embodiments, 0.0001 ml to 10 ml are delivered to a subject. In some embodiments, the number of AAV particles administered to a subject may be on the order ranging from 10⁶-10¹⁴ vg/kg, or any values therebetween, such as for example, about 10¹, 10², 10³, 10⁴, 10⁵, 10⁶, 10⁷, 10⁸, 10⁹, 10¹⁰, 10¹¹, 10¹², 10¹³, or 10¹⁴ vgs/kg. In some embodiments, when a first AAV particle comprising a first polynucleotide as described herein and second AAV particle comprising a second polynucleotide as described herein are administered, the amount administered is the same for both particles. In some embodiments, when a first AAV particle comprising a first polynucleotide as described herein and second AAV particle comprising a second polynucleotide as described herein are administered, the amount administered is different for each particle.

If desired, AAV particles may be administered in combination with other agents or treatments as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The AAV particles may thus be delivered along with various other agents or treatments as required in the particular instance.

### Use of Blocking Anti-TSHR Antibodies to Treat Thyroid Disorders

The present disclosure is based, at least in part, on the realization that blocking anti-TSHR antibodies may be delivered in a continuous manner using rAAV, e.g., rAAV8, to effectively block the stimulating effects of TSAbs or TSH on TSHR, thereby blocking or reducing the synthesis of thyroid hormone. By blocking or reducing the synthesis of thyroid hormone, the presently described methods and compositions for rAAV-based delivery of an “effective amount” of a blocking anti-TSHR antibody (e.g., K1-70) that may be used to treat thyroid diseases, including Graves' disease, Graves' orbitopathy, and thyroid cancer without the requirement of repeated administrations.

In some embodiments, “an effective amount” refers to the amount of each active agent (e.g. blocking anti-TSHR antibody) required to confer therapeutic effect on the subject, either alone or in combination with one or more other active agents (e.g., other hyperthyroidism medications). Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration (e.g., injection of a composition comprising an rAAV encoding a blocking antibody of interest) may be determined and adjusted over the course of therapy.

An exemplary dosing regimen comprises administering an initial dose of about 2 mg/kg, followed by a weekly maintenance dose of about 1 mg/kg of the antibody, or followed by a maintenance dose of about 1 mg/kg every other week. The dosing achieved may refer to the concentration of the delivered payload by the rAAV delivery vehicle. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of AAV particles may be an amount of the particles that are capable of transferring an expression construct to a host organ, tissue, or cell. A therapeutically acceptable amount may be an amount that is capable of treating a disease, e.g., hyperthyroid disorder. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

### Subject

Aspects of the disclosure relate to methods for use with a subject (e.g., a mammal) having a thyroid disorder, such as Graves' disease. In some embodiments, a mammalian subject is human or a non-human primate. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other examples of subjects include domesticated animals such as dogs and cats; livestock such as horses, cattle, pigs, sheep, goats, and chickens; and other animals such as mice, rats, guinea pigs, and hamsters.

In some embodiments, a subject is senile. In some embodiments, a subject is old (e.g., greater than 40, greater than 50, greater than 60, greater than 70, greater than 80, or greater than 90 years of age). In some embodiments, a subject suffers from or is at risk of developing a disease or disorder that involves the thyroid or thyroid malfunction. Examples of such diseases include Graves' disease, hyperthyroidism, autoimmune disorders which affect the thyroid, as well as signs and symptoms of people experiencing hyperthyroidism, including nervousness, insomnia, high heart rate, eye disease and anxiety.

Without further elaboration, it is believed that one skilled in the art can, based on the above description, utilize the present disclosure to its fullest extent. The following specific embodiments are, therefore, to be construed as merely illustrative, and not limiting of the remainder of the disclosure in any way whatsoever. All publications cited herein are incorporated by reference for the purposes or subject matter referenced herein.

### EXAMPLES

### Example 1: Vector-Based Therapy for Thyroid Disease

Background

Thyroid-related disease is associated with either inadequate production (i.e., hypothyroidism) or overproduction (i.e., hyperthyroidism) of thyroid hormones. Both types of disease are relatively common afflictions of man and animals. Hyperthyroidism, in particular, results from heightened secretion of thyroid hormones. In most species, this condition is less common than hypothyroidism.

Graves' disease (GD) is the most common form of hyperthyroidism and effects 1 in 10,000 children and 1 in 1,000 adults (1-4). An autoimmune disorder, GD is due to stimulation of the thyroid-stimulating hormone receptor (TSHR) by receptor stimulating antibodies or immunoglobulins (TSAbs or TSI) (1-4). Antibody stimulation of the TSHR triggers increased levels of cAMP within the thyroid follicular epithelial cells, leading to increase production of thyroid hormone and gland growth (1-4).

Current treatment of GD ranges from antithyroid medication therapy, radioactive iodine, to surgery (1-3). Two anti-thyroid medications are available, including methimazole and propylthiouracil (1-3), which despite their widespread use, have associated toxicity (2, 5). Available data also show that in children and adults, prolonged treatment with antithyroid medications only increases the risk of lasting remission by 20 to 30 percent at best (2, 5). Thus, most patients need definitive therapy either in the form of surgery or radioactive iodine, each of which have additional associated risks (1-4).

At the present, few alternative forms of therapy for GD are available or in development (6-7). Thus, there is considerable interest the developing novel treatments for hyperthyroidism. The inventors have herein proposed and described the development of a novel form of treatment for GD involving recombinant adeno-associated virus (rAAV) mediated expression of an antibody that blocks the TSHR. This approach will be of benefit to other related conditions as well, including Graves' orbitopathy and thyroid cancer.

For example, like in GD, TSAbs also play a role in the pathogenesis of GO (8-11). Thus, the ability to block TSAb action may also have merit for treating this condition as well. Approximately 40% of adult patients with GD will also develop GO, which will be severe in about 5% of individuals (8-11). The pathogenesis of this condition is believed to involve immune-mediated interaction with TSHRs located on retrobulbar tissue (8, 9). Even if the hyperthyroid state is treated definitively by surgery or radioactive iodine, GO may progress, and in some cases require immune modulation and or surgery (8-11).

Gene therapy-based delivery of blocking anti-TSHR antibodies may also be useful in the treatment of thyroid cancer. Thyroid cancer in children and adults involves differentiated (papillary and follicular) and undifferentiated (anaplastic) forms (12, 13). Treatment of thyroid cancer typically involves thyroidectomy and lymph node dissection, often followed by radioactive iodine (12, 13). In addition, it is believed that endogenous TSH stimulates the growth of thyroid cancer cells (12, 13). Thus, suppression of TSH action through supra-physiologic doses of levothyroxine is instituted (10, 11); however, there may be limitations to the use of relatively high doses of levothyroxine, including adverse effects related to a prolonged hyperthyroid state (3, 14). Thus, in addition to our novel therapeutic having a potential role in the treatment of GD and GO, delivery of blocking anti-TSHR antibodies by gene therapy-based vectors to continuously block TSH action may also be an effective approach in thyroid cancer treatment.

The TSH receptor consists of alpha and beta subunits with the alpha subunit containing the binding site for TSH (15-18). TsAbs play a key role in the pathogenesis of GD and GO by binding to the alpha subunit (17-18). Monoclonal antibodies have been identified that stimulate the TSH receptor and include the antibodies M22 and MS-1 (17-22). In addition to antibodies that stimulate the TSHR (and which increases synthesis of thyroid hormone), thyroid blocking antibodies (TBAbs) may bind to this receptor and either have blocking or no functional effects. Monoclonal TBAbs have been isolated and include KSAb1 and KSAb2 (23, 24). Of note, the TBAb K1-70 is being tested in human clinical trials for safety and tolerability in Graves' disease through IM injections (see https://clinicaltrials.gov/ct2/show/NCT02904330). The K1-70 antibody is available in the public domain (50). However, infusion of antibodies, antibody fragments, or peptides that potentially block TSAbs, such as K1-70, last only for a short period of time and require repeated administration (6, 7). Improved methods of delivering clinically significant levels of thyroid-blocking antibodies which would not require repeated administrations would represent a significant advancement in the art for treating thyroid disorders, such as Graves' disease, Graves' orbitopathy, and thyroid cancer.

The inventors have developed a novel recombinant gene therapy vector based on rAAV for delivering clinically significant levels of thyroid-blocking antibodies.

Wild-type AAV is a non-pathogenic virus that has been used for gene therapy (25). The use of rAAV to express antibodies that target viruses has been demonstrated for the human immunodeficiency virus (HIV) and other viruses (33, 34). Although vector-based immunotherapy has been proposed for infectious diseases (47-50), such approaches to target antibody-mediated conditions (such as thyroid disorders) are limited, making this project unique.

Therapeutic Construct

The inventors have developed a new rAAV to deliver antibodies that block thyroid TSHR activation as a novel therapeutic for thyroid diseases, such as Graves' disease, Graves' orbitopathy, and thyroid cancer.

Preliminary Studies

There is considerable interest in the development of novel therapeutic approaches for the treatment of GD and GO. In addition, the ability to continuously block TSH action may have potential merit in the treatment of differentiated thyroid cancer. Data is presented below which helps support this therapeutic approach.

Identification of the Thyroid Blocking Immunoglobulins.

It was proposed that expression of TBAbs (thyroid-blocking antibodies) will inhibit TSHR activation. To identify such immunoglobulins, a number of commercially available antisera that target the TSHR were screened. For antibody screening, a cell line that expresses TSHR was used. These Human Embryonic Kidney (HEK-293) cells have been stably transfected with the human TSH receptor (HEK-TSHR). Stimulation of the TSHR by TSH or TSAbs was assessed by measuring levels of cAMP (since stimulated TSHR activates adenylate cyclase thereby producing cAMP) (47).

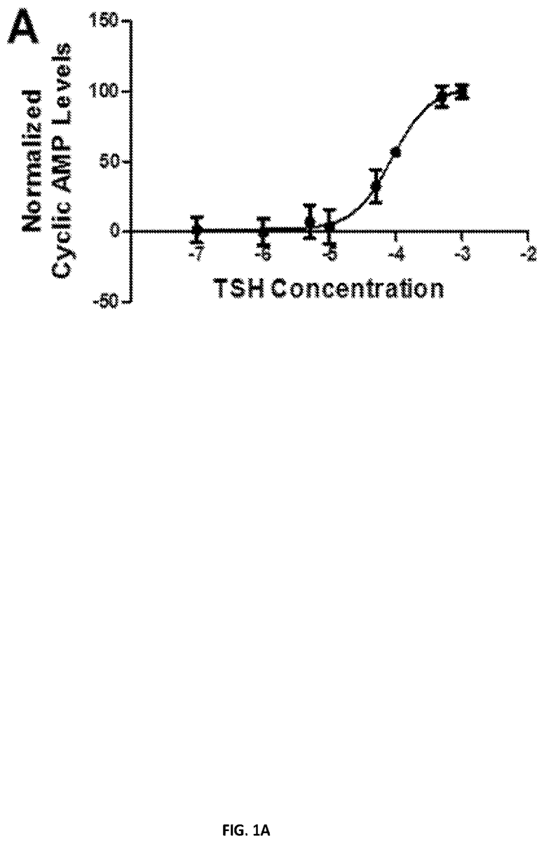

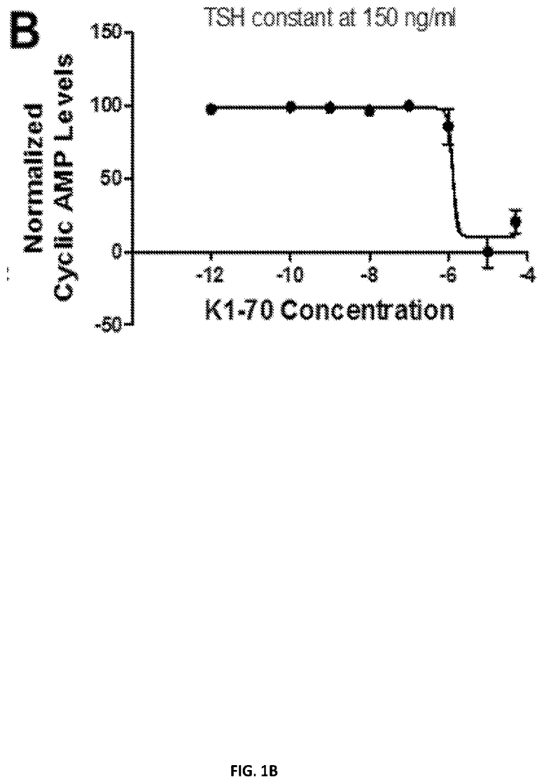

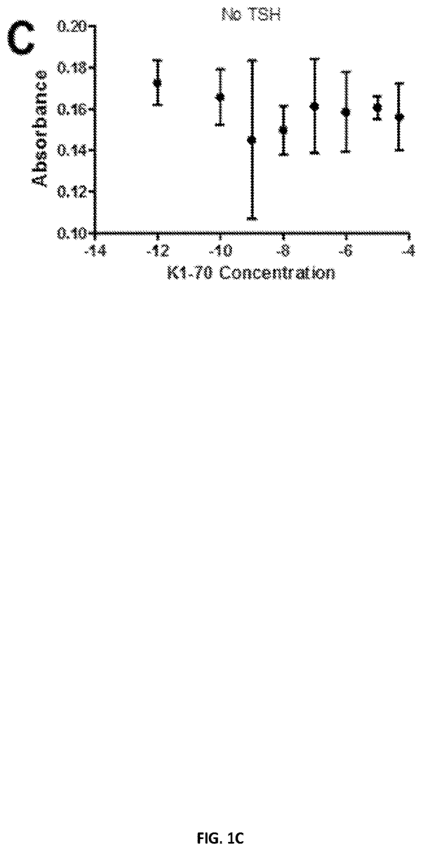

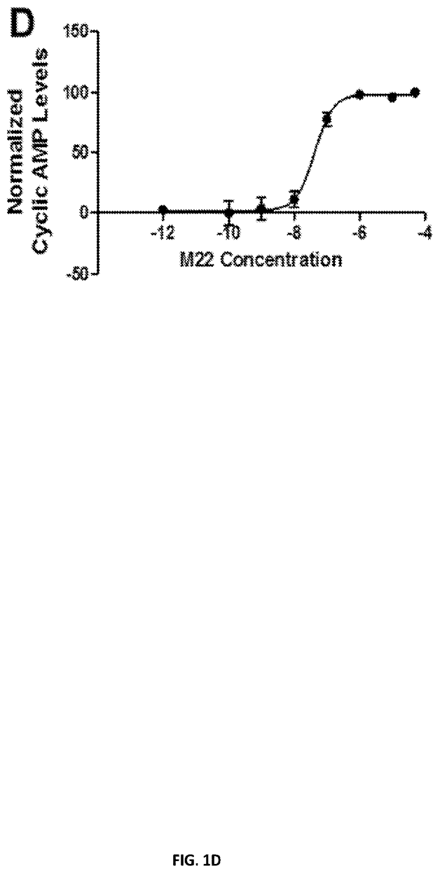

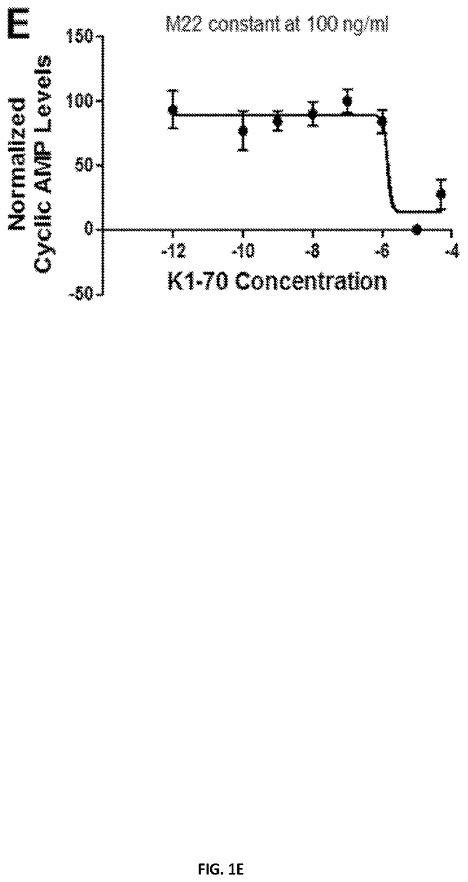

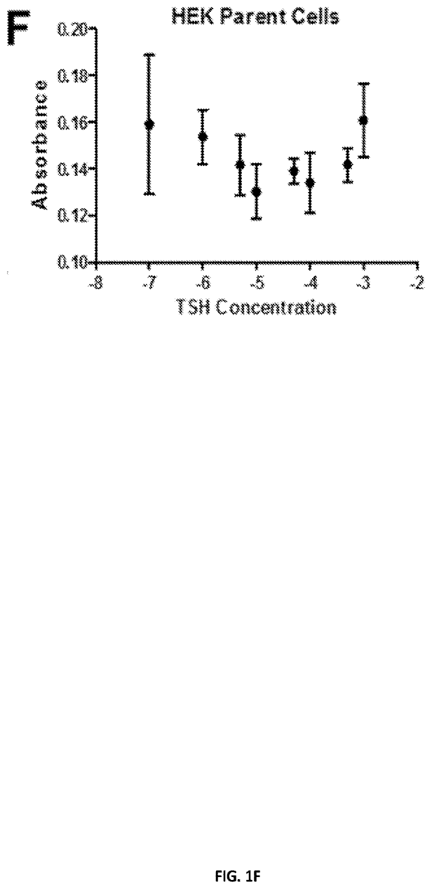

Treating HEK-TSHR cells with recombinant TSH (1 to 1000 ng/ml) resulted in a dose-response showing increased cAMP levels with increasing concentrations of TSH (FIG. 1A; EC50 167.6 ng/ml±50.6 ng/ml, N=3). To test TBAb K1-70, the cells were treated with TSH (150 ng/ml) and K1-70 (100 μg/ml to 10 μg/ml). It was found that K1-70 inhibited TSH activity and reduced cAMP levels in a dose-dependent manner (FIG. 1B; IC50 for K1-70, 1.18±0.04 μg/ml, N=3). Next, the TSAb M22 was tested and it was observed that this antibody increased cAMP levels in a dose-dependent manner (EC50 of 42.5 μg/ml) (FIG. 1D).

Finally, K1-70 was tested to determine if it inhibits the activity of the TSAb M22. The cells were treated with 100 ng/ml of M22 with a varied concentration of K1-70. It was observed that increasing concentrations of K1-70 blocked M22 stimulation (IC50 of 1.38 μg/ml, FIG. 1E). As negative controls, the parent HEK-293 cell line that does not express human TSHR was tested and observed to have no response to TSH treatment (FIG. 1F). It was also observed that K1-70 alone does not stimulate cAMP production (FIG. 1C). Thus, these data show that the TBAb K1-70 blocks TSHR activation by TSH and the TSAb M22.

Identification of rAAV Vector to Express Immunoglobulins.

Essential for gene therapeutic approaches is the identification of rAAV vectors that express immunoglobulins. There are approximately 6 well-characterized AAV serotypes available for routine laboratory testing in gene therapy (48, 49). For the proposed approach, AAV vectors that express proteins in muscle or liver were considered. Of the different serotypes, rAAV8 was selected.

A vector construct was designed to express both the heavy and light chain of the K1-70 thyroid blocking antibody. First, the K1-70 gene sequence was synthesized and cloned into a basic pUC57vector by GenScript (Piscataway, N.J.). This clone consisted of the following structure: HSP-VH-CH-F2A-LSP-VL-CL, with human IgG1 heavy chain (HC) signal peptide (HSP), K1-70 HC variable region (VH), IgG1 HC constant region (CH), F2A cleavage site, lambda-1 light chain (LC) signal peptide (LSP), K1-70 LC variable region (VL), and human LC constant region (CL). Next, this K1-70 sequence was cloned into a single stranded AAV8 vector with a chicken beta actin promoter, poly-A tail, an HA tag, and inverted terminal repeats (ITR) to result in the final vector, AAV8-K170.

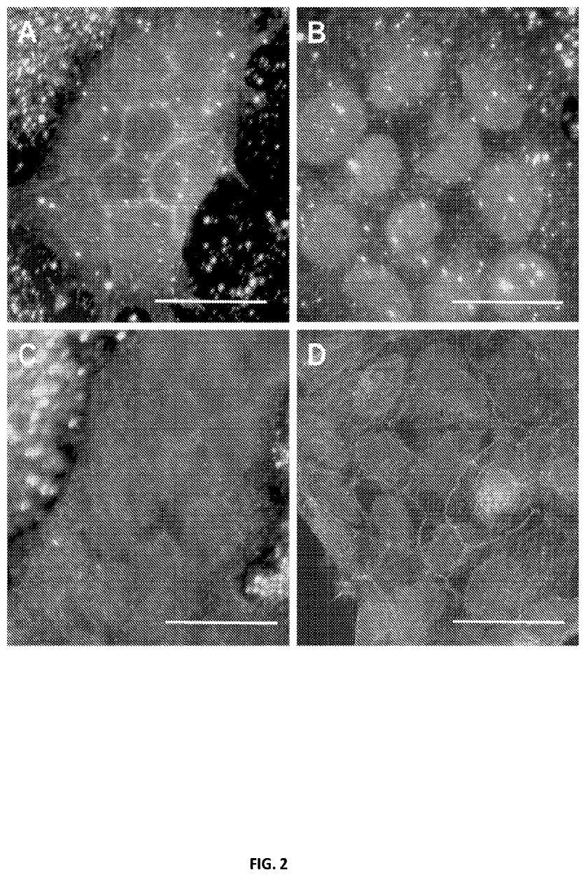

To test if AAV8-K170 is able to express antibodies that bind to the TSHR, HEK-293 cells were transfected with 1 μg AAV8-K1-70 vector and 2-4 μl of the transfection reagent, Lipofectamine 3000 (ThermoFisher), for each well of a 12-well tissue culture dish. Three days after transfection, cells were collected in PBS plus complete mini, a protease inhibitor cocktail (Roche Diagnostics, Mannheim, Germany). Following sonication of the samples, protein concentration of the lysates were determined with Pierce BCA Protein Assay Kit (Thermofisher). For immunostaining with the cell lysate, HEK-293 cells, that express human TSHR, were plated on coverslips in a 12 well tissue culture plate. Cells were grown overnight, fixed in 4% PFA, and permeabilized with 0.5% Triton X-100 in PBS. After blocking in 2% BSA and 2% goat serum, 25 ug of cell lysate was used as a primary antibody, followed by a secondary antibody, Alexa Fluor goat anti-human IgG (Invitrogen) (FIG. 2A). Cell lysate labeling of HEK-TSHR cells appears to be specific, as no labeling was observed in the no primary antibody control (FIG. 2B) or when used on the parent cell line that does not express TSHR (FIG. 2C). The commercially available K1-70 antibody used in the above cAMP assays was also used as a primary antibody (FIG. 2D), which displayed similar staining patterns as the AAV8-K1-70 vector cell lysates. K1-70 immunostaining at cell-cell junctions was observed in both the commercial K1-70 and cell lysate from AAV8-K1-70 transfected cells, indicating that it is binding to cell surface receptors (FIG. 2).

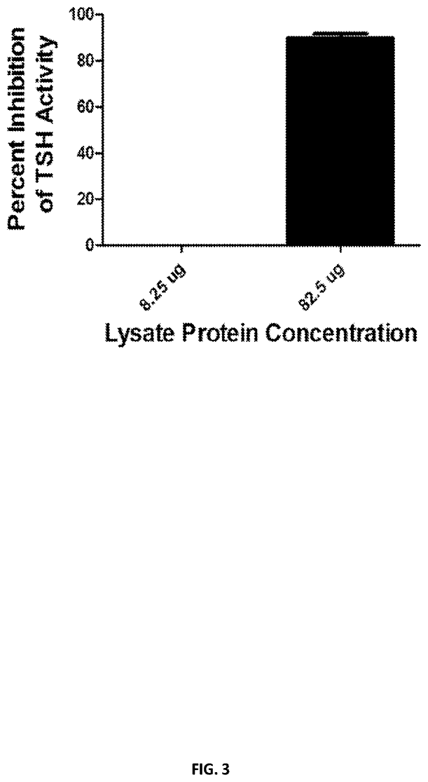

To determine if AAV8-K170 is able to produce TSHR blocking antibodies, HEK-TSHR cells were treated directly with cell lysate collected from HEK-293 cells expressing the AAV8-K1-70 vector, as described above. Next, performed a cAMP assay was performed as described above. A full TSH curve was shown as in FIG. 1A as a standard, and tested 2 different concentrations of cell lysate (FIG. 3). The cell lysate at 1/10 dilution, 8.25 μg total cell lysate protein per well, was unable to block TSH action and thus was unable to reduce the amount of cAMP produced with 150 ng/ml of TSH stimulation. However, when the cell lysate was used at full strength (82.5 μg a well) to treat the HEK-TSHR cells, an 89.6%±3.4% (N=3, P≤0.05) inhibition of TSH activity was observed (FIG. 3). These results are consistent with the K1-70 inhibition curve observed with the commercial K1-70, as inhibition is minimal until it reaches a high enough concentration and it switches

To determine if AAV8-K170 produces the intended TSHR blocking antibodies, the HEK-TSHR cells were treated directly with cell lysate from the HEK-293 cells expressing the AAV8-K1-70 vector, as described above. Next, cAMP assays were performed as described above, with a fixed TSH concentration of 150 ug/ml, and tested 2 different concentrations of cell lysate (FIG. 3). When the whole cell lysate was used at a 1:10 dilution (8.25 μg total cell lysate protein per 250 μl well), it did not block TSH stimulation of cAMP. However, when the cell lysate was tested at a concentration of 82.5 μg per well, an 89.6%±3.4% (N=3, P≤0.05) was observed.

This example demonstrates that cell lysates from HEK-293 cells expressing the AAV8-K1-70 vector are able to inhibit TSH activity leading to reduced levels of cAMP in response to TSH stimulation (FIG. 3). The results suggest that the rAAV-TBAb vector will target and block TSHR activation, leading to inhibition of thyroid hormone production and other TSHR-mediated events.

### REFERENCES

Each of the following references are herein incorporated by reference in their entireties as part of this disclosure.
1. Ross D S, Burch H B, Cooper D S, et al. 2016 American Thyroid Association Guidelines for Diagnosis and Management of Hyperthyroidism and Other Causes of Thyrotoxicosis. Thyroid: official journal of the American Thyroid Association 2016; 26:1343-421.
2. Rivkees S A. Controversies in the management of Graves” disease in children. J Endocrinol Invest 2016; 39:1247-57.
3. Rivkees S A. Thyroid disorders in children and adolescents. In: Sperling M A, ed. Pediatric Endocrinology Fourth edition. United States: Saunders; 2014:444-70.
4. Ueda D. Normal volume of the thyroid gland in children. J Clin Ultrasound 1990; 18:455-62.
5. Rivkees S A, Mattison D R. Ending propylthiouracil-induced liver failure in children. N Engl J Med 2009; 360:1574-5.
6. Fassbender J, Holthoff H P, Li Z, Ungerer M. Therapeutic Effects of Short Cyclic and Combined Epitope Peptides in a Long-Term Model of Graves” Disease and Orbitopathy. Thyroid: official journal of the American Thyroid Association 2019; 29:258-67.
7. Holthoff H P, Li Z, Fassbender J, et al. Cyclic Peptides for Effective Treatment in a Long-Term Model of Graves' Disease and Orbitopathy in Female Mice. Endocrinology 2017; 158:2376-90.
8. Bahn R S. Current Insights into the Pathogenesis of Graves” Ophthalmopathy. Horm Metab Res 2015; 47:773-8.
9. Bartalena L, Baldeschi L, Boboridis K, et al. The 2016 European Thyroid Association/European Group on Graves” Orbitopathy Guidelines for the Management of Graves” Orbitopathy. Eur Thyroid J 2016; 5:9-26.
10. Nagayama Y, Nakahara M, Abiru N. Animal models of Graves” disease and Graves” orbitopathy. Curr Opin Endocrinol Diabetes Obes 2015; 22:381-6.
11. Sabatino J, Donati S, Bartalena L. Graves” Orbitopathy: do not give it for granted. Endocrine 2018; 62:731-2.
12. Francis G L, Waguespack S G, Bauer A J, et al. Management Guidelines for Children with Thyroid Nodules and Differentiated Thyroid Cancer. Thyroid: official journal of the American Thyroid Association 2015; 25:716-59.
13. Haugen B R, Alexander E K, Bible K C, et al. 2015 American Thyroid Association Management Guidelines for Adult Patients with Thyroid Nodules and Differentiated Thyroid Cancer: The American Thyroid Association Guidelines Task Force on Thyroid Nodules and Differentiated Thyroid Cancer. Thyroid: official journal of the American Thyroid Association 2016; 26:1-133.
14. Rivkees S A, Mazzaferri E L, Verburg F A, et al. The treatment of differentiated thyroid cancer in children: emphasis on surgical approach and radioactive iodine therapy. Endocr Rev 2011; 32:798-826.
15. Parmentier M, Libert F, Maenhaut C, et al. Molecular cloning of the thyrotropin receptor. Science 1989; 246:1620-2.
16. Rees Smith B, McLachlan S M, Furmaniak J. Autoantibodies to the thyrotropin receptor. Endocr Rev 1988; 9:106-21.
17. Ungerer M, Fabbender J, Holthoff H P. Antigen-specific therapy of Graves' disease and orbitopathy by induction of tolerance. Front Biosci (Landmark Ed) 2018; 23:2044-52.
18. Ungerer M, Fassbender J, Li Z, Munch G, Holthoff H P. Review of Mouse Models of Graves” Disease and Orbitopathy-Novel Treatment by Induction of Tolerance. Clin Rev Allergy Immunol 2017; 52:182-93.
19. Chen C R, Hubbard P A, Salazar L M, McLachlan S M, Murali R, Rapoport B. Crystal structure of a TSH receptor monoclonal antibody: insight into Graves” disease pathogenesis. Mol Endocrinol 2015; 29:99-107.
20. Furmaniak J, Sanders J, Nunez Miguel R, Rees Smith B. Mechanisms of Action of TSHR Autoantibodies. Horm Metab Res 2015; 47:735-52.
21. Liu C, Hermsen D, Domberg J, et al. Comparison of M22-based ELISA and human-TSH-receptor-based luminescence assay for the measurement of thyrotropin receptor antibodies in patients with thyroid diseases. Horm Metab Res 2008; 40:479-83.
22. Roggenbuck J J, Veiczi M, Conrad K, et al. A novel third-generation TSH receptor antibody (TRAb) enzyme-linked immunosorbent assay based on a murine monoclonal TSH receptor-binding antibody. Immunol Res 2019.
23. Gilbert J A, Gianoukakis A G, Salehi S, et al. Monoclonal pathogenic antibodies to the thyroid-stimulating hormone receptor in Graves” disease with potent thyroid-stimulating activity but differential blocking activity activate multiple signaling pathways. J Immunol 2006; 176:5084-92.
24. Marcinkowski P, Hoyer I, Specker E, et al. A New Highly Thyrotropin Receptor-Selective Small-Molecule Antagonist with Potential for the Treatment of Graves” Orbitopathy. Thyroid: official journal of the American Thyroid Association 2019; 29:111-23.
25. Kotin R M. Prospects for the use of adeno-associated virus as a vector for human gene therapy. Human gene therapy 1994; 5:793-801.
26. Gao G, Vandenberghe L H, Wilson J M. New recombinant serotypes of AAV vectors. Current gene therapy 2005; 5:285-97.
27. Hollenbaugh J A, Gee P, Baker J, et al. Host factor SAMHD1 restricts DNA viruses in non-dividing myeloid cells. PLoS pathogens 2013; 9:e1003481.
28. McCarty D M, Monahan P E, Samulski R J. Self-complementary recombinant adeno-associated virus (scAAV) vectors promote efficient transduction independently of DNA synthesis. Gene Ther 2001; 8:1248-54.
29. Flotte T R, Carter B J. Adeno-associated virus vectors for gene therapy. Gene Ther 1995; 2:357-62.
30. Asokan A, Schaffer D V, Samulski R J. The AAV vector toolkit: poised at the clinical crossroads. Molecular therapy: the journal of the American Society of Gene Therapy 2012; 20:699-708.
31. Ferrari F K, Xiao X, McCarty D, Samulski R J. New developments in the generation of Ad-free, high-titer rAAV gene therapy vectors. Nature medicine 1997; 3:1295-7.
32. Muller O J, Kaul F, Weitzman M D, et al. Random peptide libraries displayed on adeno-associated virus to select for targeted gene therapy vectors. Nature biotechnology 2003; 21:1040-6.
33. Balazs A B, Chen J, Hong C M, Rao D S, Yang L, Baltimore D. Antibody-based protection against HIV infection by vectored immunoprophylaxis. Nature 2011; 481:81-4.
34. Balazs A B, Ouyang Y, Hong C M, et al. Vectored immunoprophylaxis protects humanized mice from mucosal HIV transmission. Nature medicine 2014; 20:296-300.
35. Choi V W, McCarty D M, Samulski R J. AAV hybrid serotypes: improved vectors for gene delivery. Current gene therapy 2005; 5:299-310.
36. Srivastava A, Carter B J. AAV Infection: Protection from Cancer. Human gene therapy 2017; 28:323-7.
37. Sen D, Balakrishnan B, Gabriel N, et al. Improved adeno-associated virus (AAV) serotype 1 and 5 vectors for gene therapy. Sci Rep 2013; 3:1832.
38. Hareendran S, Balakrishnan B, Sen D, Kumar S, Srivastava A, Jayandharan G R. Adeno-associated virus (AAV) vectors in gene therapy: immune challenges and strategies to circumvent them. Rev Med Virol 2013; 23:399-413.
39. Ponnazhagan S, Mukherjee P, Yoder M C, et al. Adeno-associated virus 2-mediated gene transfer in vivo: organ-tropism and expression of transduced sequences in mice. Gene 1997; 190:203-10.
40. Shimojo N, Kohno Y, Yamaguchi K, et al. Induction of Graves'-like disease in mice by immunization with fibroblasts transfected with the thyrotropin receptor and a class II molecule. Proc Natl Acad Sci USA 1996; 93:11074-9.
41. McLachlan S M, Nagayama Y, Rapoport B. Insight into Graves” hyperthyroidism from animal models. Endocr Rev 2005; 26:800-32.
42. Clement S C, Kremer LCM, Verburg F A, et al. Balancing the benefits and harms of thyroid cancer surveillance in survivors of Childhood, adolescent and young adult cancer: Recommendations from the international Late Effects of Childhood Cancer Guideline Harmonization Group in collaboration with the PanCareSurFup Consortium. Cancer Treat Rev 2018; 63:28-39.
43. Martino A T, Basner-Tschakarjan E, Markusic D M, et al. Engineered AAV vector minimizes in vivo targeting of transduced hepatocytes by capsid-specific CD8+ T cells. Blood 2013; 121:2224-33.
44. Nathwani A C, Tuddenham E G, Rangarajan S, et al. Adenovirus-associated virus vector-mediated gene transfer in hemophilia B. N Engl J Med 2011; 365:2357-65.
45. Zhong L, Li W, Li Y, et al. Evaluation of primitive murine hematopoietic stem and progenitor cell transduction in vitro and in vivo by recombinant adeno-associated virus vector serotypes 1 through 5. Human gene therapy 2006; 17:321-33.
46. AGTC Lands the First Billion-Dollar Deal for a U F Startup. http://research.ufl.edu/otl/agtc-lands-the-first-billion-dollar-deal-for-a-uf-startup.html. 2015.
47. Kleinau G, Neumann S, Gruters A, Krude H, Biebermann H. Novel insights on thyroid-stimulating hormone receptor signal transduction. Endocr Rev 2013; 34:691-724.
48. Wu Z, Asokan A, Samulski R J. Adeno-associated virus serotypes: vector toolkit for human gene therapy. Molecular therapy: the journal of the American Society of Gene Therapy 2006; 14:316-27.
49. Zincarelli C, Soltys S, Rengo G, Rabinowitz J E. Analysis of AAV serotypes 1-9 mediated gene expression and tropism in mice after systemic injection. Molecular therapy: the journal of the American Society of Gene Therapy 2008; 16:1073-80.
50. Furmaniak J, Sanders J, Young S, et al. In vivo effects of a human thyroid-stimulating monoclonal autoantibody (M22) and a human thyroid-blocking autoantibody (K1-70). Auto Immun Highlights 2012; 3:19-25.
51. Sanders P, Young S, Sanders J, et al. Crystal structure of the TSH receptor (TSHR) bound to a blocking-type TSHR autoantibody. J Mol Endocrinol 2011; 46:81-99.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A recombinant adeno-associated virus (rAAV) particle comprising a nucleic acid molecule that encodes a blocking thyroid-stimulating hormone receptor antibody (anti-TSHR antibody) or fragment thereof.

2. The rAAV particle of claim 1, wherein the nucleic acid molecule comprises a promoter operably linked to an expression cassette.

3. The rAAV particle of claim 2, wherein the promoter is a truncated chimeric CMV-chicken β-actin (smCBA) promoter.

4. The rAAV particle of claim 2, wherein the expression cassette comprises in a 5′-to-3′ direction a first sequence encoding a heavy chain of the blocking anti-TSHR antibody, a second sequence encoding a self-cleaving site, and a third sequence encoding a light chain of the blocking anti-TSHR antibody.

5. The rAAV particle of claim 2, wherein the expression cassette comprises in a 5′-to-3′ direction a first sequence encoding a light chain of the blocking anti-TSHR antibody, a second sequence encoding a self-cleaving site, and a third sequence encoding a heavy chain of the blocking anti-TSHR antibody.

6. The rAAV particle of claim 4 or 5, wherein the self-cleaving site is F2A cleavage site.

7. The rAAV particle of claim 4 or 5, wherein the first sequence and the third sequence are each preceded by a signal sequence.

8. The rAAV particle of claim 2, wherein the nucleic acid molecule further comprises a poly-A tail sequence.

9. The rAAV particle of claim 2, wherein the nucleic acid molecule further comprises an HA sequence.

10. The rAAV particle of claim 2, wherein the nucleic acid molecule further comprises inverted terminal repeat (ITR) sequences at the 5′ and 3′ ends of the expression cassette.

11. The rAAV particle of claim 1, wherein the rAAV is serotype 1, 2, 3, 4, 5, 6, 7, 8, 9, 10, 11, 12, 13, 2/1, 2/5, 2/8, 2/9, 3/1, 3/5, 3/8, or 3/9.

12. The rAAV particle of claim 1, wherein the rAAV is serotype 8.

13. The rAAV particle of claim 1, wherein the blocking anti-TSHR antibody is K1-70.

14. The rAAV particle of claim 13, wherein the K1-70 has a heavy chain of SEQ ID NO: 1 or a sequence having at least 90% sequence identity to SEQ ID NO: 1 and a light chain of SEQ ID NO: 2 or a sequence having at least 90% sequence identity to SEQ ID NO: 2.

15. The rAAV particle of any one of the preceding claims, wherein the nucleic acid further encodes a detectable molecule.

16. The rAAV particle of claim 6, wherein the P2A tag becomes cleaved such that the heavy chain and the light chain are expressed as separate molecules.

17. A composition comprising a plurality of the rAAV particles of any one of the preceding claims, and a pharmaceutically acceptable carrier.

18. A kit comprising the compositions of claim 17, and instructions for using the composition.

19. A method for treating a hyperthyroid disorder, comprising administering an effective amount of the composition of claim 17 to a subject.

20. The method of claim 19, wherein the subject is human.

21. The method of claim 19, wherein the hyperthyroid disorder is Graves' disease.

22. The method of claim 19, wherein the hyperthyroid disorder is Graves' orbitopathy.

23. The method of claim 19, wherein the hyperthyroid disorder is thyroid cancer.

24. The method of claim 19, wherein the anti-TSHR antibody blocks activation of a thyroid-stimulating hormone receptor (TSHR) by a thyroid-stimulating hormone (TSH) and a thyroid-stimulating antibody (TSAb).

25. The method of claim 19, wherein the anti-TSHR antibody inhibits TSH production.

26. A method of blocking the activation of a TSHR cell, comprising a plurality of the rAAV particles of any one of claim 1-16, and a pharmaceutically acceptable carrier.

27. A method of blocking the activation of a TSHR cell, comprising administering an effective amount of the composition of claim 17 to a subject.

28. The method of claim 27, wherein the subject is human.

29. Use of the rAAV particle of claim 1 for delivering the antibody to the TSHR.

30. Use of the rAAV particle of claim 1 for treating or diagnosing a hyperthyroid disorder.


SEQUENCE LISTING (26 sequences)

SEQ ID NO: 1 - protein, 221 aa, Homo sapiens
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 2 - protein, 214 aa, Homo sapiens
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTE

SEQ ID NO: 3 - protein, 118 aa, Mus musculus
ELVFTQPPAIMSASPGEKVTISCSASSSVSYMCWFQQKPGSSPKPWIYRTSNLASGVPARFSGSGSGTSYSLTISSMEAEDAATYYCQQYHSYPLTFGAGTKLELKRADAAPTVSIFP

SEQ ID NO: 4 - protein, 125 aa, Mus musculus
EVQLLESGPELVKPGASVKMSCKASGYTFTSYIIHWVKQKPGQGLEWIGYINLYNDGTNYNEKFTGKATLTSDKSSSTAYMELSSLTSEDSAVYYCAREDYYGRVADFDVWGAGTTVTVSSAKTT

SEQ ID NO: 5 - protein, 764 aa, Homo sapiens
MRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHEMRQGLHNMEDVYELIENSHLTPKKQGQISEEYMQTVL

SEQ ID NO: 6 - protein, 20 aa, Artificial Sequence
  Synthetic
GGSGGGSGGGGSGGGSGGGG

SEQ ID NO: 7 - DNA, 60 bp, Artificial Sequence
  Synthetic
ggcggcagcggcggcggcagcggcggcggcggcagcggcggcggcagcggcggcggcggc

SEQ ID NO: 8 - protein, 21 aa, Artificial Sequence
  Synthetic
METDTLLLWVLLLWVPGSTGD

SEQ ID NO: 9 - DNA, 63 bp, Artificial Sequence
  Synthetic
atggagaccgacaccctgctgctgtgggtgctgctgctgtgggtgcccggcagcaccggcgac

SEQ ID NO: 10 - protein, 22 aa, Artificial Sequence
  Synthetic
MAAPVPWACCAVLAAAAAVVYA

SEQ ID NO: 11 - protein, 19 aa, Artificial Sequence
  Synthetic
MQALVLLLCIGALLGHSSC

SEQ ID NO: 12 - protein, 37 aa, Artificial Sequence
  Synthetic
MWKWILTHCASAFPHLPGCCCCCFLLLFLVSSVPVTC

SEQ ID NO: 13 - protein, 20 aa, Artificial Sequence
  Synthetic
MRTLACLLLLGCGYLAHVLA

SEQ ID NO: 14 - protein, 30 aa, Artificial Sequence
  Synthetic
MAPSLSPGPAALRRAPQLLLLLLAAECALA

SEQ ID NO: 15 - protein, 23 aa, Artificial Sequence
  Synthetic
MTPWLGLIVLLGSWSLGDWGAEA

SEQ ID NO: 16 - protein, 26 aa, Artificial Sequence
  Synthetic
MNFLLSWVHWSLALLLYLHHAKWSQA

SEQ ID NO: 17 - protein, 29 aa, Artificial Sequence
  Synthetic
MPPSGLRLLLLLLPLLWLLVLTPGRPAAG

SEQ ID NO: 18 - protein, 18 aa, Artificial Sequence
  Synthetic
MRLLAKIICLMLWAICVA

SEQ ID NO: 19 - protein, 20 aa, Artificial Sequence
  Synthetic
MTSKLAVALLAAFLISAALC

SEQ ID NO: 20 - protein, 23 aa, Artificial Sequence
  Synthetic
MKVSAALLCLLLIAATFIPQGLA

SEQ ID NO: 21 - protein, 19 aa, Artificial Sequence
  Synthetic
MKLWDVVAVCLVLLHTASA

SEQ ID NO: 22 - protein, 22 aa, Artificial Sequence
  Synthetic
AAAATNFSLLKQAGDVEENPGP

SEQ ID NO: 23 - DNA, 66 bp, Artificial Sequence
  Synthetic
gccgccgccgccaccaacttcagcctgctgaagcaggccggcgacgtggaggagaaccccggcccc

SEQ ID NO: 24 - protein, 237 aa, Artificial Sequence
  Synthetic
MSKGEELFTGVVPILVELDGDVNGHKFSVSGEGEGDATYGKVNGHKFSVSGEGEGDATYGKTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELY

SEQ ID NO: 25 - DNA, 717 bp, Artificial Sequence
  Synthetic
atgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgaccaccctgacctacggcgtgcagtgcttcagccgctaccccgaccacatgaagcagcacgacttcttcaagagcgccatgcccgagggctacgtgcaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggccacaagctgcgcatcgagctgaagggcatcgacttcaaggaggacggcaacatcctgggccacaagctgaaggtgaacttcaagatccgccacaacatcgaggacggcagcgtgcagctggccgaccactaccagcagaacacccccatcggcgacggccccgtgctgctgcccgacaaccactacctgagcacccagagcgccctgagcaaggaccccaacgagaagcgcgaccacatggtgctgctggagttcgtgaccgccgccggcatcacccacggcatggacgagctgtacaagtaa

SEQ ID NO: 26 - DNA, 959 bp, Artificial Sequence
  Synthetic
gaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattc